(19) United States
(12) Patent Application Publication
(10) Pub. No.: US 2015/0359595 A1
(43) Pub. Date: Dec. 17, 2015

(54) HYBRID CATHETER APPARATUS AND METHODS

(71) Applicant: EXIMO MEDICAL LTD., Rehovot (IL)

(72) Inventors: Ilan Ben Oren, Modi'in (IL); Yoel Zabar, Ness Ziona (IL)

(21) Appl. No.: 14/764,180

(22) PCT Filed: Jan. 31, 2014
(86) PCT No.: PCT/IB2014/058688

§ 371 Date: Jul. 29, 2015

Related U.S. Application Data
(60) Provisional application No. 61/758,830, filed on Jan. 31, 2013.

(51) Int. Cl. A61B 18/24; A61B 18/14; A61B 17/32

(52) U.S. Cl. CPC A61B 18/24; A61B 17/320016; A61B 18/1492; A61B 2018/0088

(57) ABSTRACT

A hybrid catheter device for performing cutting action on a region of a tissue, using sequential laser and mechanical cutting processes, and incorporating an acoustic sensor on the end of the device, such that if a pulsed laser beam is used for the laser cutting, the absorption of that beam in the tissue being cut can provide information about the progress of the cut by means of the opto-acoustic effect. Other hybrid catheter devices incorporate blunt protrusions on the end of said device, but having sharp lateral edges, such that rotation of the catheter device generates mechanical cutting action in the tissue. The blunt protrusion ends prevents uncontrolled cutting in the forward motion. Other hybrid catheter devices enable controlled incisions into an organ wall, such as the duodenum, and held in place by means of an inflatable balloon. A dual-wavelength nail fungus treatment hybrid catheter is also shown.

### FIELD OF THE INVENTION

The invention relates to the use of hybrid catheters in various cutting and therapeutic procedures, using a combination of laser or electrical discharge energy operating in conjunction with mechanical cutting, especially for treatment in vascular vessels.

### BACKGROUND OF THE INVENTION

There is an important need for enabling solutions for resection of lesions or deposits in a variety of applications where the target is in a delicate organ that is subject to perforation, especially with regard to flat lesions where physical handling of the shape of the lesion presents difficulties because of their low profile and proximity to the walls of the organ in which they are situated. The following is a brief survey of some types of treatments that may require the sensitive removal of tissue without damage to the healthy underlying tissue base.

Flat lesions especially present a significant challenge in gastroenterology. Removing of sessile and flat polyps, which may be associated with high risk for malignancy, requires, in most cases, usage of different techniques than those used for removing common polyps. These techniques may lead to the referral of patients to surgery instead of removal by the gastroenterologist. Other challenging lesions are nonpolypoid colorectal neoplasms (NP-CRNs). Barrett's esophagus is another common chronic condition. The prevalence in the U.S. population is estimated to be in the range of 1-2% of the adult population. Barrett's esophagus condition may lead to violent esophageal cancer, which is said to result in over 12,000 deaths per year in the U.S. alone and around 100,000 in China.

Management of Barrette's Esophagus.

Barrett's Esophagus (BE) is a common disorder that is a major risk factor for esophagus cancer. The prevalence of the disorder is estimated to be in the range of 1-2%. See Ronkainen J, Aro P, Storskrubb T, et al. (December 2005) “Prevalence of Barrett's esophagus in the general population: an endoscopic study”, Gastroenterology 129 (6): 1825-31. The range of severity can vary from early stage to different grades of dysplasia to cancer. Prior attempts to manage this condition with Argon coagulation yielded controversial results. Alternative methods are based on RF ablation (RFA) (Halo® System), photodynamic therapy (PDT), cryo, thermal therapy or surgery as endoscopic mucosal resection (EMR). No method resulted in wide clinical acceptance that can enable routine use in a broad population instead of “waiting and watching” in early stages in the disease and specific therapies including esophagus resection in more sever conditions.

Furthermore, as no single technique has been established as the preferred method, a combination of techniques is used in certain cases. For example, there may be a consensus that RFA is useful for patients with BE and high-grade dysplasia (HGD), BE and intramucosal carcinoma as an adjunct to endoscopic mucosal resection (EMR). The use of RFA for BE with low-grade dysplasia (LGD) or intestinal metaplasia is not clearly established, see David E. Fleischer Virender K. Sharma, Interventional and Therapeutic Gastrointestinal Endoscopy. Front Gastrointest Res. Basel, Karger, 2010, vol 27, pp 140-146. On the other hand, EMR sometimes does not allow removing all of the Barrett's lining but can be successful in removing a small cancer or a localized area of high-grade dysplasia. Because it does not remove all of the Barrett's lining, the Barrett's lining left behind can develop other areas of high-grade dysplasia or cancer. Therefore, EMR is sometimes combined with photodynamic therapy in an attempt to remove remaining Barrett's tissue or with RF ablation. Conversely, several photodynamic therapy studies have also reported that a few patients have a situation in which the Barrett's lining does not completely go away but is still there, underneath the new normal-appearing squamous lining (and detected when biopsy is performed that shows that small areas of Barrett's lining are still there underneath the new squamous lining.) In such a case, another course of treatment with another technique might be beneficial.

Complications of the current available techniques include perforations (making a hole in the esophagus), bleeding, strictures, light sensitivity in PDT and even death.

Removal of Challenging Lesions in Intestine and Stomach.

Some of the polyps and adenomas (benign tumors) detected with an increasing percent in colonoscopy, with different imaging techniques, do not have a conventional “pedunculated” shape. Polyps that are not attached to the surface by a narrow elongated stalk are called sessile. Other polyps are not significantly elevated from the adjacent mucosa are called flat. Accordingly, the removal of large sessile and flat colorectal is more difficult than removal of pedunculated polyps and in many cases require using special endoscopy techniques to avoid perforation.

These lesions may be associated with high clinical risk. The incidence of cancer with submucosal invasion appears to be higher in flat-type lateral spreading tumors.

Endoscopic Mucosal Resection (EMR) is becoming the standard technique for resection of large sessile and flat colorectal lesions. For the more challenging lesions, Endoscopic Submucosal Dissection (ESD) can be used. ESD can be performed using a viscous injection solution for sustained submucosal lifting, a diathermy knife, and a plastic hood to help retract the polyp as it is dissected away from the muscularis propria.

Although these techniques are feasible anywhere in the colon, currently these techniques are technically challenging and time consuming and ESD carries a relatively high rate of major complication. Laser ablation is usually not perceived as an adequate solution for this application, as there is a need to assure adequate (i.e. complete) removal of pathological tissue and preferably to collect resected samples for histological analysis.

There is thus an unmet need in the art for devices, systems and methods that would allow efficient and effective vascular interventions as well as removal of challenging lesions in the gastroenterology (GI) track (mainly in colon and stomach) and Barrett's esophagus management.

Removal of Challenging Lesions in Gynecology and Urology.

There is a need for effective and safe tools for removal of pathological tissue in gynecology (cervical, uterus and conditions such as endometriosis) and urology (urinary bladder, prostate), wherein the depth of resection can be controlled while risk of perforation and bleeding is minimized.

Modulation of Signals Associated by Passage of Food in the GI Tract and Modification of Physiology—

Dissections and Incision in the GI track in the management of diabetes type, insulin resistance and obesity. Furthermore, there is a need for endoscopy or catheter based minimally invasive enabling technologies that can provide at least some of the benefits of bariatric surgery. Several studies have shown that bariatric surgery can lead to remission of diabetes type 2 or improvement in its management, including improvements in insulin resistance. This is especially true for specific surgery procedures such as those that bypass the duodenum. There are approximately 300 million patients that suffer from diabetes type 2 world wile and the cost and side effects of surgery limits its utility as a viable solution for such a large population.

Removal of Nails and Pathogens in the Management of Nail Fungus.

There are a variety of solutions based on the use of lasers for the management of nail fungus. These treatments suffer from two limitations, as the nail is not removed it takes a few months until an appropriate response to treatment can be evaluated without and with addition suffering from a limited response to treatment of the technique.

Laser and Mechanical Based Solutions for Angioplasty, Atherectomy and Thrombectomy:

Peripheral and arterial vascular diseases are also a common problem which may directly lead to morbidity and death. In the U.S. alone it is estimated that over 4 million people suffer from peripheral artery disease which, in severe cases, is treated with surgery or even amputation. The current state of the art in laser ablation technology for vascular intervention is based on use of an Excimer lasers with dedicated catheters such as Spectranetics' CVX-300® laser and TURBO-Booster® catheter. These technologies are described, for example, in U.S. Pat. Nos. 6,673,064, 7,811,281 and 7,572,254. Due to technical and safety considerations, the excimer laser used, generally, is often a Xenon Chloride laser operative at 308 nm with pulse widths in the range of 100 nanoseconds. These technologies are not ideal and have some limitations. For example, when dealing with heavy calcified plaques, there is a risk of perforation and damage from debris/plaque fragments. Therefore, the procedure requires a complex, large and costly system and the length of the procedure is quite significant in a manner that seems to limit its wide clinical utility. In addition, the technique had difficulties in treatment of large vessels such as SFA (Superficial Femoral Artery) which is very important in management of peripheral artery disease (PAD) wherein vessels larger than 4-5 mm in diameter and long lesions have to be treated.

One of the reasons for the length of the process is that even one of the most advanced solutions, combining the TURBO-Booster® and the TURBO Laser Elite catheters, may require a number of steps starting with atherectomy to create an initial pilot channel through the whole lesion, for example using the laser catheter alone, and only at later stages the laser catheter is loaded into the introducer sheath. The use of the catheter is based on several passages, each after rotation of the catheter. See Schwarzwälder U, Zeller T, Tech Vasc Intery Radiol. 2010 March; 13(1):43-53.

Additional limitations of this solution include ineffective removal of arterial debris and high risk of artery walls injury, as mentioned, for example, in U.S. Pat. No. 6,962,585:

“An Excimer Laser Coronary Angioplasty system and procedure offered by Spectranetics of Colorado Springs, Colo., involves the insertion into an artery of a laser catheter containing a bundle of optical fibers and a stent with a guide wire. The laser catheter is advanced in the artery until the guide wire crosses a blockage, at which time bursts of ultraviolet (cool) laser light is transmitted through the fiber optic fibers to open a hole in the blockage. Thereafter, an x-ray contrast dye is injected into the blood stream to determine the extent to which the artery has been opened. This procedure does not remove substantial amounts of blockage because ultra violet radiation is too cool to melt the blockage. Rather, a hole is blasted through the blockage to accommodate the admission of a stent. While the catherization system includes a filter, the filter is not sufficient to catch all debris which may flow downstream. Such prior systems have failed because they have not effectively removed arterial blockage from the artery walls, and have not effectively removed arterial debris from the artery once the arterial blockage has been dislodged. In addition, such prior systems have not adequately protected the artery walls from physical or thermal injury. Further, many of the prior art devices embody numerous parts which tend to fail or shatter in a high temperature/high vacuum environment.” (id, p. 1, In. 19).

An alternative approach using IR laser for thermal heating of a tip used to cut the plaques to be removed with suction is disclosed in U.S. Pat. No. 6,962,585. This approach may suffer from the limitations and risk involved with plaque removal based on non-selective heating. The approach proposed in this case is to use arterial guards in the outer part of the catheter that may limit the passage of the catheter and avoid getting closer to the walls. Other attempts to use thermal effects include a hybrid thermal probe, wherein most of the laser energy (Argon or Nd:YAG) is used to heat the hot tip in the catheter, and part of it escapes as laser light. Clinical results were not satisfactory to enable routine clinical use.

Additional prior approaches include use of a laser to core the plaque and use of mechanical means to “ingest” and remove the plaque. See, for example, U.S. Pat. No. 4,979,939. In Canadian Patent No. 1,326,800 a fiber is introduced to create an opening through which the distal rotary is introduced and the second fiber is used to vaporize the material collected by the blade. U.S. Patent Application Publication No. 2010/0125253 discloses a dual tip catheter for treating chronic total occlusions through which a fiber may be introduced.

In view of the complexity and limitations of the laser based technologies, the systems based on excimer laser have had limited spread in clinical use, and alternative mechanical methods for atherectomy have been developed, for example, wherein the plaques are “shaved” (the EV3 product), “drilled” (the Pathway product) or “sanded” with a rotating diamond coated brush (the CSI product). Each of these techniques may often suffer from inherent limitations such as procedure length, injury to the blood vessels, difficulty in dealing with calcified plaques in certain cases and, on the contrary, dealing with soft plaque (see Schwarzwälder U, Zeller T, Tech Vasc Intery Radiol. 2010 March; 13(1):43-53) or discarding of plaque material into the blood stream.

It should be noted that it is assumed by experts in the area that injury of healthy tissue and the characteristics of the tissue after plaque is removed may affect the healing (and initial hyperplasia) and the rate of restenosis which seems to be a limitation of some of the above-mentioned techniques. Furthermore, in view of the limited capabilities to remove plaque with many prior techniques, their current utility is limited mainly for use in conjunction with low pressure balloon angioplasty used after plaque is partially removed. The balloon then opens the vessel with the remaining plaque material.

The need to deal with complete or partial blockage in vessels applies also to artificial grafts, such as those implanted in the legs for bypass, for hemodialyisis access and more.

In-Stent—Restenosis.

It is known in the art that in a significant percentage of the patients that underwent stent implantation, restenosis occurs within a few years after implantation. This is a major issue with bare-metal stents (BMS) and even introduction of drug eluting stents (DES) that show a robust decrease of restenosis still does not completely solve the problem.

Acute Blockage of Vessels.

There is also a need for tools that quickly open blood vessels in patients that suffer from ischemic stroke (caused by blockage of a blood vessel) or in heart attacks with the minimal risk of perforation.

Removal of Pacemaker and Defibrillator Leads.

Presently there is a growing need to remove pacemaker and defibrillators leads in a subset of patients due to several reasons such lead fracture, abrasion of the insulation causing shorting and infections. Approximately 5 million leads are implanted worldwide and it is estimated that 4-7% will have to be removed at certain time point. It is estimated that over 100,000 leads were extracted in the US and Europe in 2010.

There are several approaches to remove transvenously introduced ICD leads. If leads have been in place for only a short period, they can frequently be removed by simple traction. After the leads are in place for long time scar tissue may withholds the leads during traction, the force applied to the leads is limited by the tensile strength of the insulation and conductor coils, therefore locking stylets and sheaths are used to enable a more forceful tension, but successful lead removal can still be very problematic when the leads is attached to sensitive tissue such myocardial wall. In severe cases lead extraction may require open surgery. The Spectranetics Excimer Laser and Cook Medical's Evolution products are currently used for lead removal using transcatheter techniques. The “debulking” of the lead using an excimer laser has yielded good clinical results but requires a large and expensive laser that does not allow wide use in any cardiology unit and a relative long learning curve is required.

There is a need for an effective and safe solution for removal of pace makers and defibrillator leads in patients. There is also a need to provide a novel solution for endoluminal interventions that overcomes the deficiencies of the prior art.

Removal of Lesions in Dermatology—

there is a need for effective removal/resection of lesions in dermatology that enable effective histology evaluation, minimize necrosis in health surroundings and can minimize spread of infection wherein relevant

In International Patent Publication No. WO 2012/114333 for “Hybrid Catheter for Vascular Intervention” and in International Patent Publication No. WO 2012/114334 for “Hybrid Catheter for Endoluminal Intervention” both having a common inventor with the inventor in the present application, and both hereby incorporated by reference, each in their entirety, there are described hybrid catheters incorporating both directed laser radiation applicators in conjunction with co-aligned mechanical cutters, in order to more efficiently resect tissue by the combined action of the two separate cutting processes.

### SUMMARY

The prior art describes devices for detaching undesired tissue from an inner wall of a body cavity, the devices having a tip section in a shape of a cylinder's sector, the tip section comprising: a plurality of optical fibers located along an inner surface of the tip section and configured to transmit laser radiation to the undesired tissue; and a cutter having a shape of a cylinder's sector located inwardly and/or outwardly to the plurality of optical fibers, wherein said cutter is configured to cut through the undesired tissue and thereby detach at least a part of the undesired tissue from the inner wall of the body cavity.

Methods have been described for detaching undesired tissue from an inner wall of a body cavity, the methods comprising: using a plurality of optical fibers, transmitting laser radiation to an area of said undesired tissue, thereby modifying said area; and cutting through said modified area using a cutter, thereby detaching at least a part of said undesired tissue.

In this disclosure, a number of additional implementations are described, which provide practical solutions to some of the problems still existent with these devices, and to propose new structures and methods of use.

There is therefore provided, a device for performing cutting action on a region of a tissue, comprising:

(i) an applicator adapted to direct energy from the distal end of the device onto the region of the tissue,
(ii) an element for performing mechanical cutting, the element being aligned such that the mechanical cutting is performed in the same regions of the tissue where the energy is applied, and
(iii) at least one acoustic sensor disposed in the distal region of the device,
wherein when the energy is pulsed, the at least one acoustic sensor can perform imaging of the tissue by detecting acoustic waves generated therein by impingement of the pulsed energy in the tissue.

In such a device, the energy may be laser energy, and the acoustic waves are generated by the photo-acoustic effect, or alternatively, the energy may be RF electrical discharge energy, and the acoustic waves are generated by the thermo-acoustic effect. In the case of laser energy, the applicator may be an array of optical fibers adapted to direct the pulsed laser energy onto the region of the tissue. The optical fibers may be arranged in a circular disposition, and the element for performing mechanical cutting may then be a circular cutter co-axially aligned with the optical fibers.

In any of the above described devices, at least one of the acoustic sensors may be an ultrasound transducer. Furthermore, the at least one acoustic sensor may be an array of acoustic sensors.

Additional implementations may involve either use of the pulsed energy also to degrade the tissue or use of a separate source of pulsed energy to degrade the tissue.

Another example implementation involves a device for performing cutting action on a region of a tissue, comprising:

(i) a circular array of optical fibers disposed at the distal end of the device such that laser emission from the optical fibers falls on the region of the tissue, and
(ii) a plurality of protrusions disposed on the distal end of the device, the protrusions having a blunt distal end face, and sharp lateral edges, such that rotation of the device causes the protrusions to generate mechanical cutting action in the region of the tissue.

In such a device, the blunt distal end faces of the protrusions may be configured such that they generate resistance to penetration of the device into the tissue when the device is pushed towards the tissue, and the lateral edges should be sufficiently sharp to cut the tissue when the device is rotated manually.

Furthermore, any such device may have a low friction coating on at least its inner wall, such that it can slide readily into the tissue. Additionally, such a device may have a rounded profile to the inner wall of its distal end, such that it can slide readily over a guide wire or a pacemaker lead or a defibrillator lead. Also, the device may have a rounded profile to the outer wall of its distal end, such that it can slide over the tissue.

Yet another implementation may involve a device for performing cutting action on a region of a tissue, comprising:

(i) an applicator adapted to direct energy from the distal end of the device onto the region of the tissue,
(ii) an element for performing mechanical cutting, the element being aligned such that the mechanical cutting is performed in the same regions of the tissue where the energy is applied,
wherein the mechanical cutting element comprises a first set of blades for detaching material from the tissue, and a second set of blades for cutting up the detached material to facilitate its removal from the region.

In such a device, at least the second set of blades may have a scooped shape for removing the detached material.

Additionally, another alternative implementation of the devices described in this disclosure may be for a device for performing incisions in the walls of a lumen of a subject, comprising:

(i) at least one side-directed hybrid catheter, the hybrid catheter comprising an ablation applicator and a mechanical cutter co-aligned with the ablation applicator, such that an incision can be made in the walls of the lumen by activation of the ablation applicator and mechanical cutter, and
(ii) a mechanism for rotating the at least one hybrid catheter around an axis generally parallel to the wall of the lumen, such that operation of the ablation applicator and the mechanical cutter enables controlled incisions to be performed in the wall of the lumen.

The ablation applicator of such a device may be either an optical ablation applicator, or an electrical discharge ablation applicator. Also, the hybrid catheter may be adapted to be attached to an endoscope for conveying the device to the location where the incisions in the walls are to be performed. The device described above may further comprise an inflatable balloon structure for pushing the device into contact with the wall of the organ when the balloon is inflated.

According to yet another implementation, in the above described device, the at least one side-directed hybrid catheter may be a plurality of side-directed hybrid catheters disposed at different positions along the length of the device, and the ablation energy may be switched sequentially between the side-directed hybrid catheters, such that the device can generate a series of incisions in a section of the wall of the organ without moving the device along the wall of the organ.

In any of the above described devices, the walls may be the inner walls of a part of the gastro-intestinal tract of the subject. Additionally, the ablation applicator and the mechanical cutter may be activated sequentially.

Furthermore, any of the above-described systems may further involve a device for performing cutting action on a region of a tissue, comprising:

(i) an optical applicator adapted to direct laser energy from the distal end of the device onto the region of the tissue,
(ii) an element for performing mechanical cutting, the element being aligned such that the mechanical cutting is performed in the same regions of the tissue where the energy is applied, and
(iii) at least one passage positioned to supply water from the distal end of the device,
wherein the laser has a wavelength which is strongly absorbed by the water, such that impingement of the laser energy on the water exiting the at least one passage generates a jet of water which impact on the tissue.

In such a device, the laser energy may have a wavelength of 2.9 microns. Furthermore, in such a device, the water jets should enable the device to operate on hard tissue, which could be either a nail tissue or a calcified lesion.

Still other example implementations involve a device for treatment of a dermatological region of a subject, the device comprising:

(i) an optical applicator for delivering a first laser emission to the dermatological region, the first laser emission being in such a range that it resects the dermatological region on which it impinges, and
(ii) a second laser emission for delivery by the optical applicator, the second laser emission being in a wavelength range that causes photo-chemical degradation of a dermatological defect in the region where the resection was achieved by the first laser emission. This device may further comprise a mechanical cutter for operating in conjunction with the resecting laser emission.

Alternatively, yet other example implementations may involve a device for treatment of a dermatological region of a subject, the device comprising:

(i) an electrode assembly for delivering an electrical discharge onto the dermatological region, such that the dermatological region is resected in the regions on which the discharge impinges, and
(ii) an optical applicator for delivering a laser emission to the dermatological region, the laser emission being in a wavelength range that causes photo-chemical degradation of a dermatological defect in the region where the resection was achieved by the electrical discharge. This device may further comprise a mechanical cutter for operating in conjunction with the resecting electrical discharge.

In either of the last two described devices, the dermatological region may be a nail of the subject, and the dermatological defect may be a fungus affecting the nail.

In addition to the above described devices, there are also described in this disclosure methods for using the above described devices for various medical procedures. Thus, there is also presented a method for using the hybrid catheter for lead extraction using the round (protruding) blades, by which method, the operator pushes forward when the laser fires and can also slightly rotate the device to enhance separation debulking.

According to another method, the hybrid catheter may be used with on-line imaging for delicate controlled tissue resection such as in Barrett's Esophagus, flat lesions in dermatology, and gynecology. In these methods, the operator controls the process based on either light based or acoustic, photoacoustic imaging to control the depth of penetration to avoid perforation and/or to detect margins of the area that needs to be removed to improve clinical efficacy

In another method, the hybrid catheter is used with an accessory that the operator can activate to stretch or attach the tissue to enhanced the action/operation of the hybrid catheter.

Additionally, this hybrid catheter may be used to resect and, either to simultaneously or immediately following the resection, to use the same or another laser light for photochemical destruction or ablation of remaining tissue. This new laser wavelength can be selected to kill pathogens such as in infected lesion, or to damage potentially abnormal cells that may remain after resection in cancer or pre-cancer lesions

In order to relate the novel implementations of the present disclosure to the devices and systems disclosed in the above mentioned International Patent Applications published as WO 2012/114333 and WO 2012/114334, details of the embodiments shown in those applications are repeated herewithin.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. It is intended that the embodiments and figures disclosed herein are to be considered illustrative rather than restrictive. The figures are listed below.

FIG. 1A shows an exemplary cylindrical tip section of a prior art hybrid catheter in perspective view;

FIG. 1B shows a prior art cylindrical tip section of a hybrid catheter in a front view;

FIG. 10 shows a prior art cylindrical tip section of a hybrid catheter inside a vessel with partial plaque blockage in a cross-sectional view;

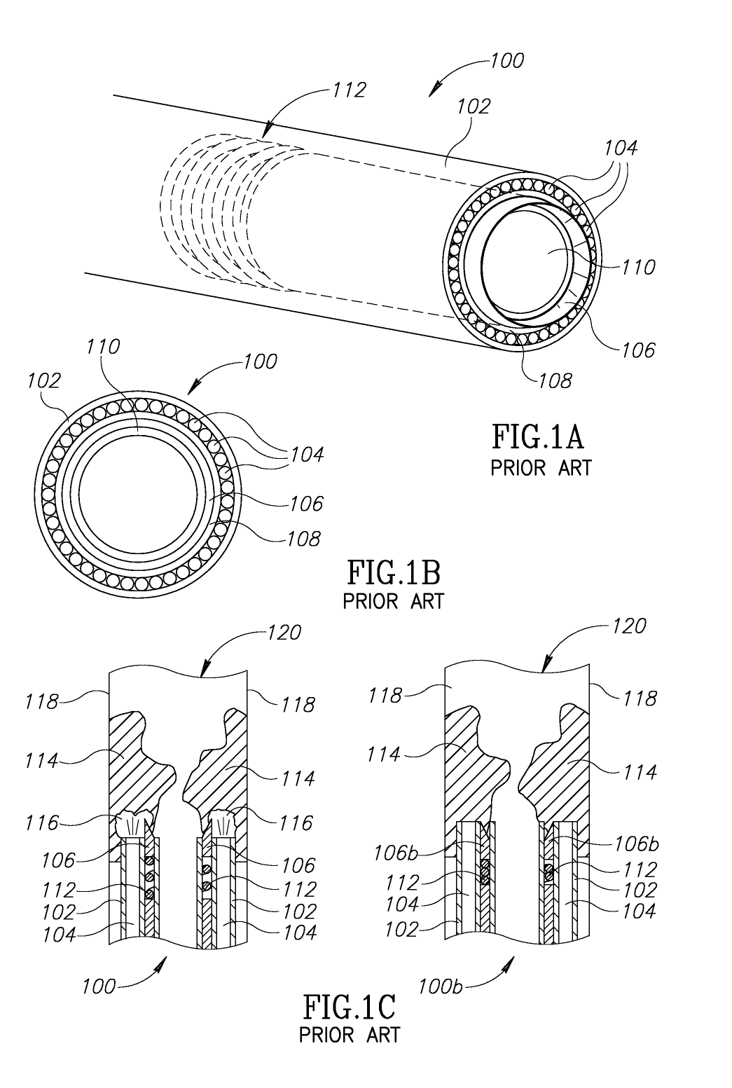

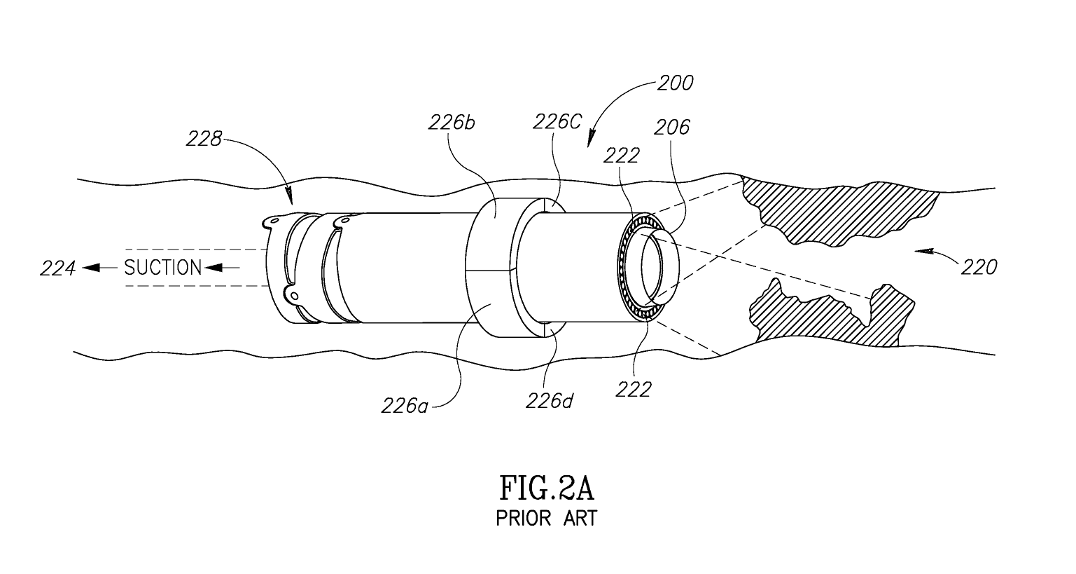

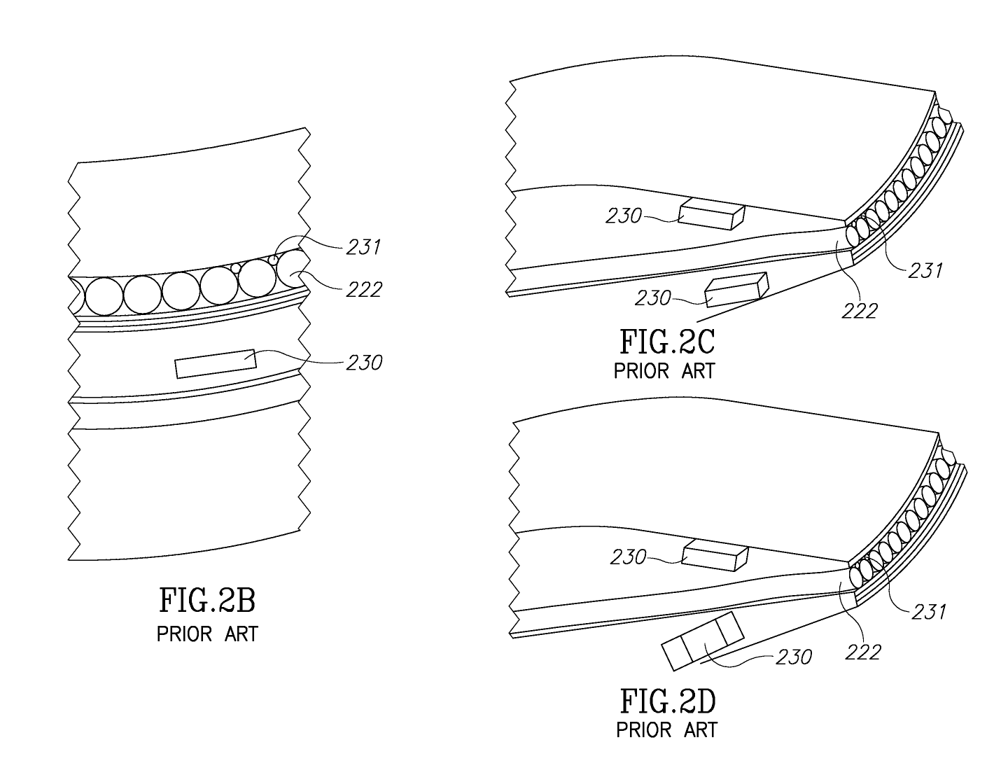

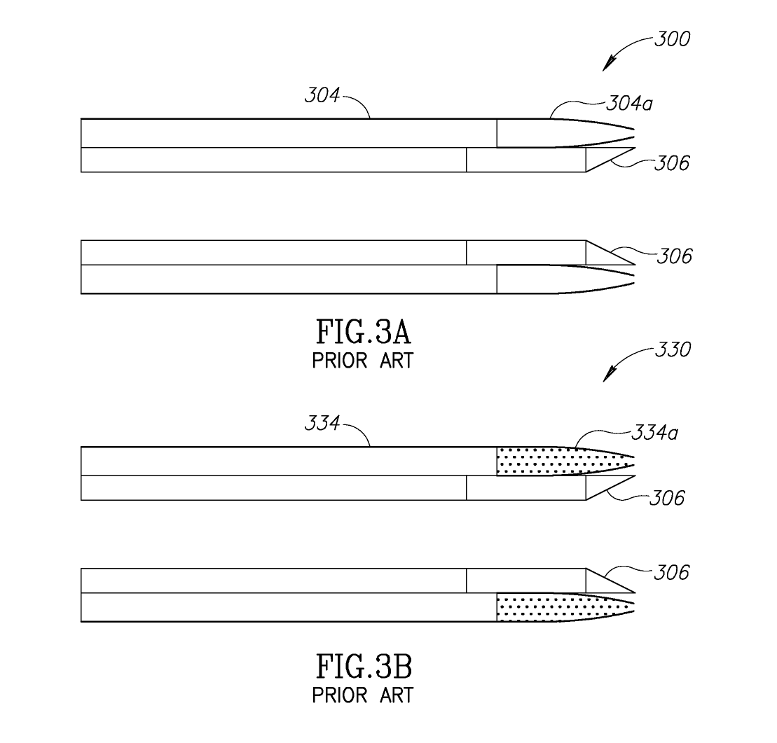

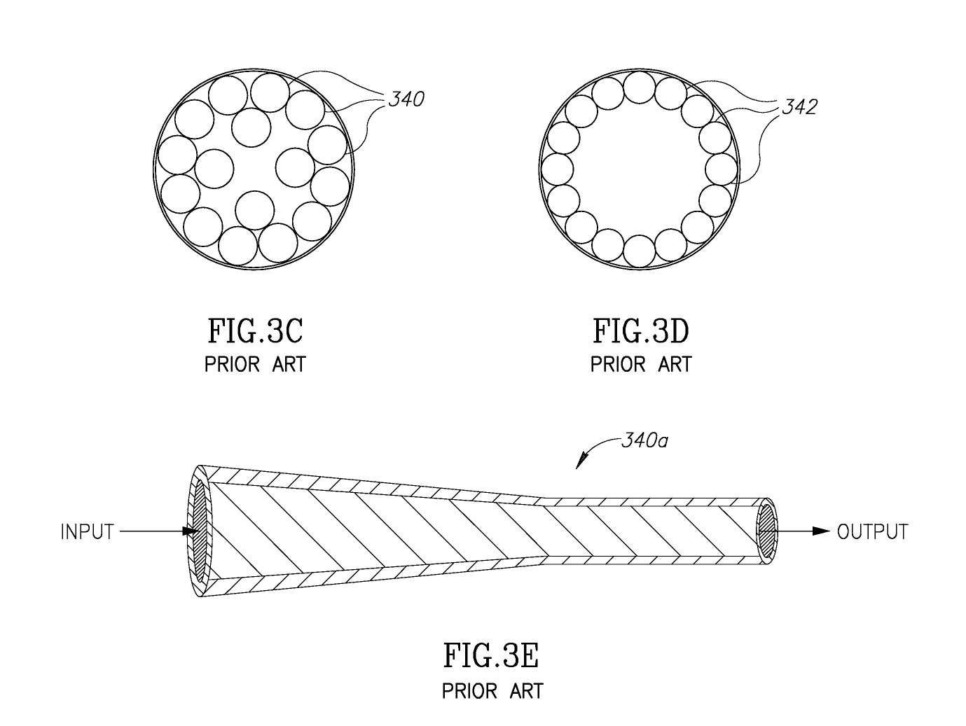

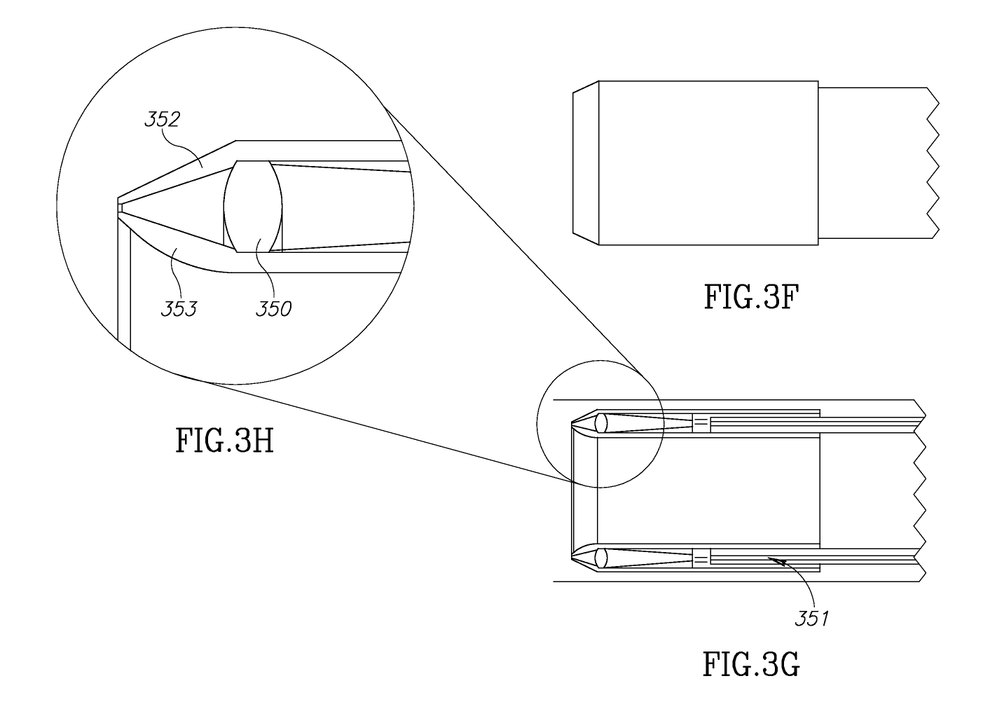

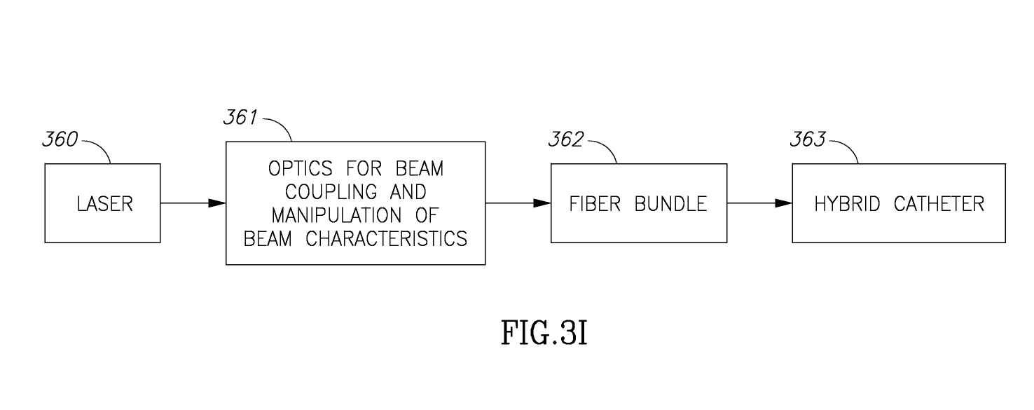

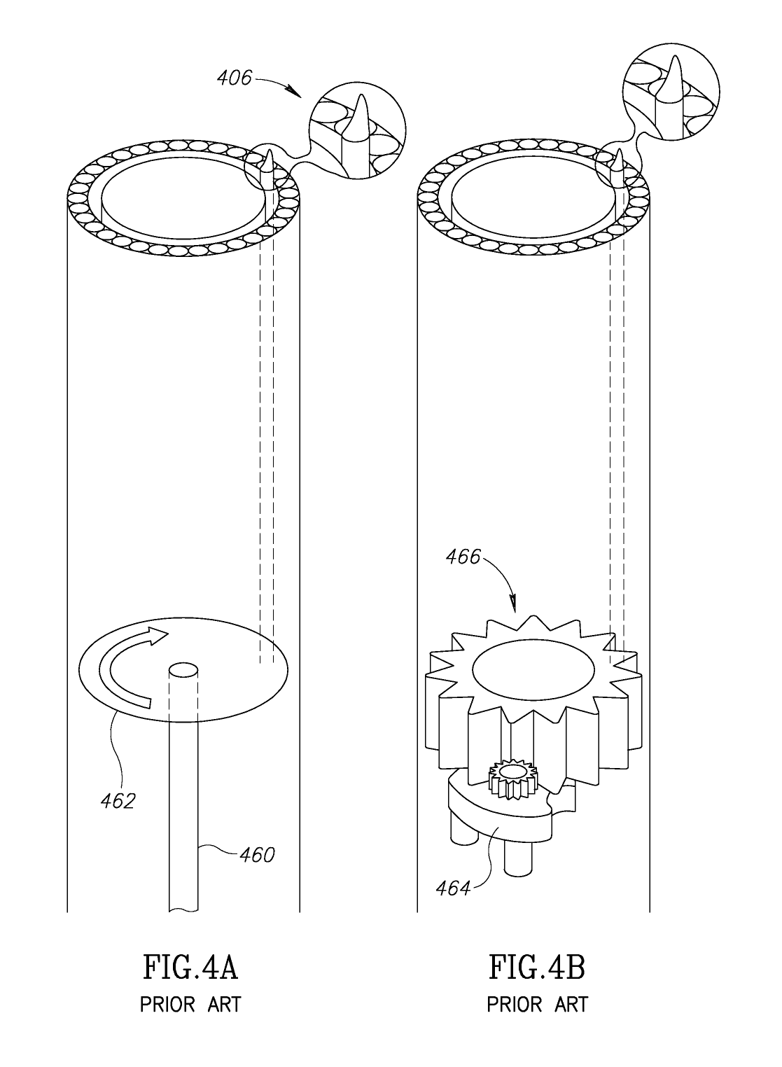

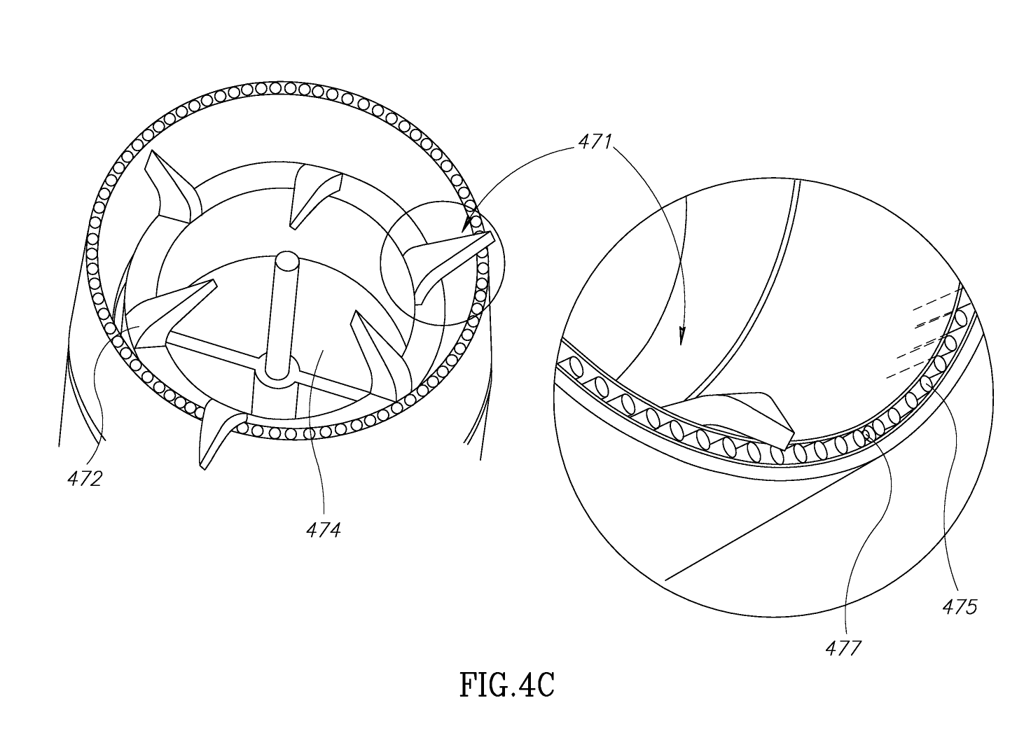

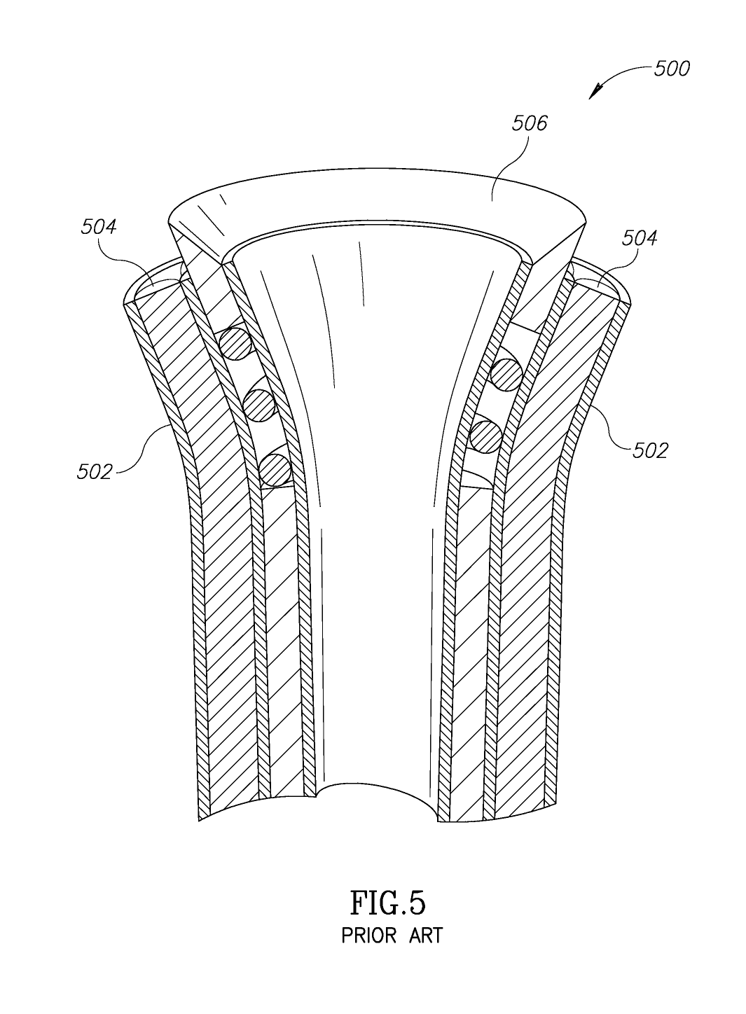

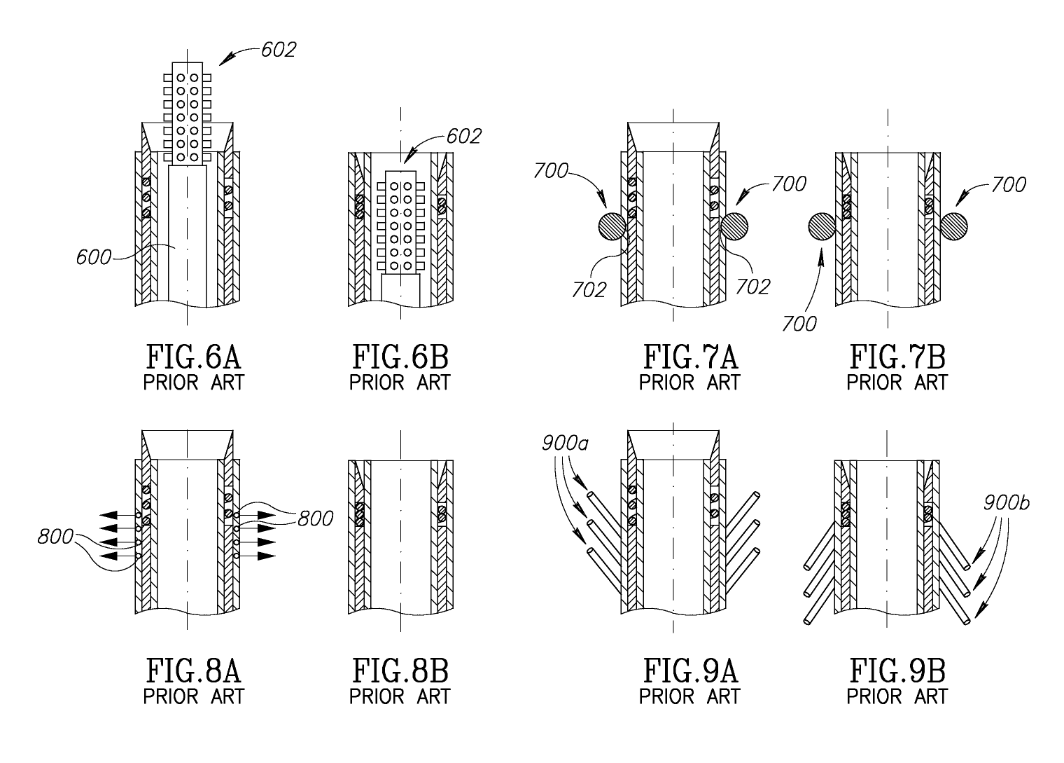

FIG. 2A shows a prior art tip section of a hybrid catheter, with one or more alterations with respect to FIGS. 1A-C;

FIGS. 2B-2D illustrate schematically other embodiments of the prior art catheters shown in FIGS. 1A and 1B, in which an acoustic sensor is incorporated into the tip of the catheter, to enable use of the photo-acoustic effect to obtain image information regarding the position of the catheter tip, and information on structures or layers deeper from the surface than is obtainable by optical imaging, including changes in the target tissue arising from the intervention, for the purpose of on-line monitoring and process control;

FIG. 3A shows a prior art tip section which includes a hollow reflective light concentrator;

FIG. 3B shows a prior art tip section which includes a solid-state light concentrating waveguide;

FIGS. 3C-3E show the usage of prior art tapered fibers;

FIGS. 3F-3H illustrate schematically a novel hybrid catheter, incorporating focusing lenses at the ends of the fibers;

FIG. 3I is a schematic block diagram of an optical delivery system for the hybrid catheters described in the present disclosure, to enable maximum possible energy delivery through the fibers;

FIGS. 4A-4B show a prior art circular-action cutter, while FIG. 4C illustrates a novel cutter with two sets of differently oriented and shaped blades;

FIG. 5 shows a cross-sectional view of a prior art expandable tip section;

FIGS. 6A-B illustrate a tube introduced through the catheter and ending with an array of nozzles or apertures;

FIGS. 7A-B illustrate the use of a roller to stain the tissue;

FIGS. 8A-B illustrate apertures built into the housing of a prior art catheter;

FIGS. 9A-B illustrate an array of tubes or needles which are used to administer the drug in a prior art implementation;

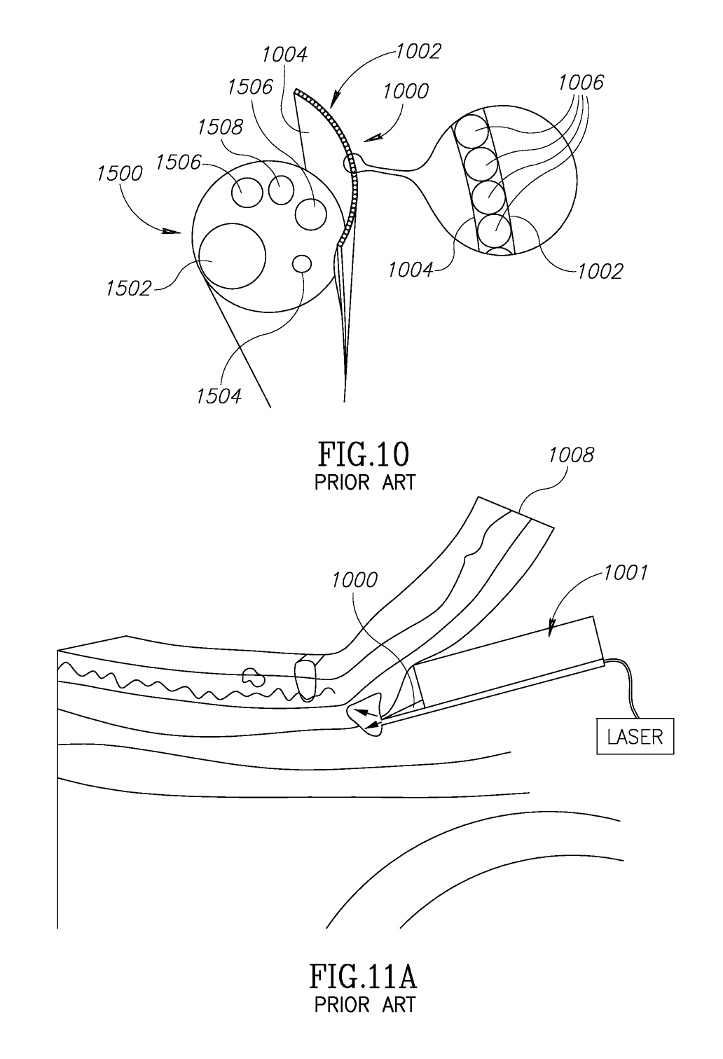

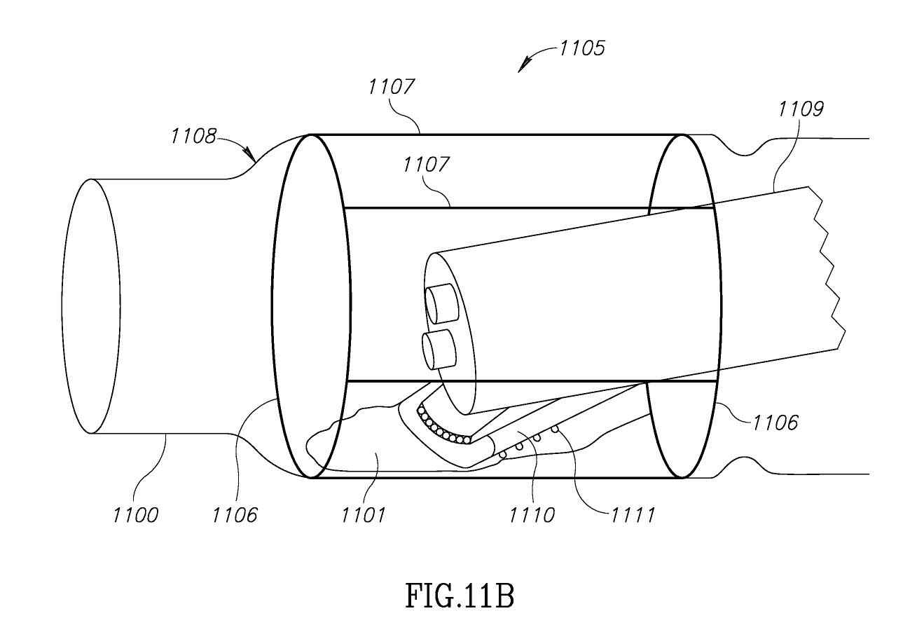

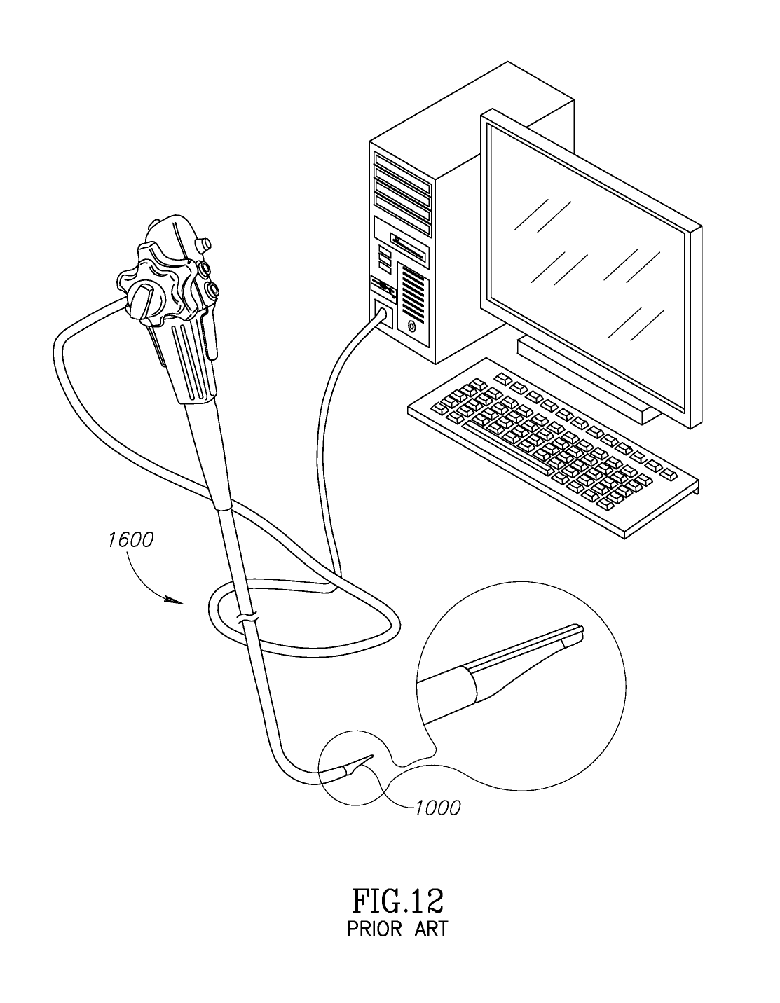

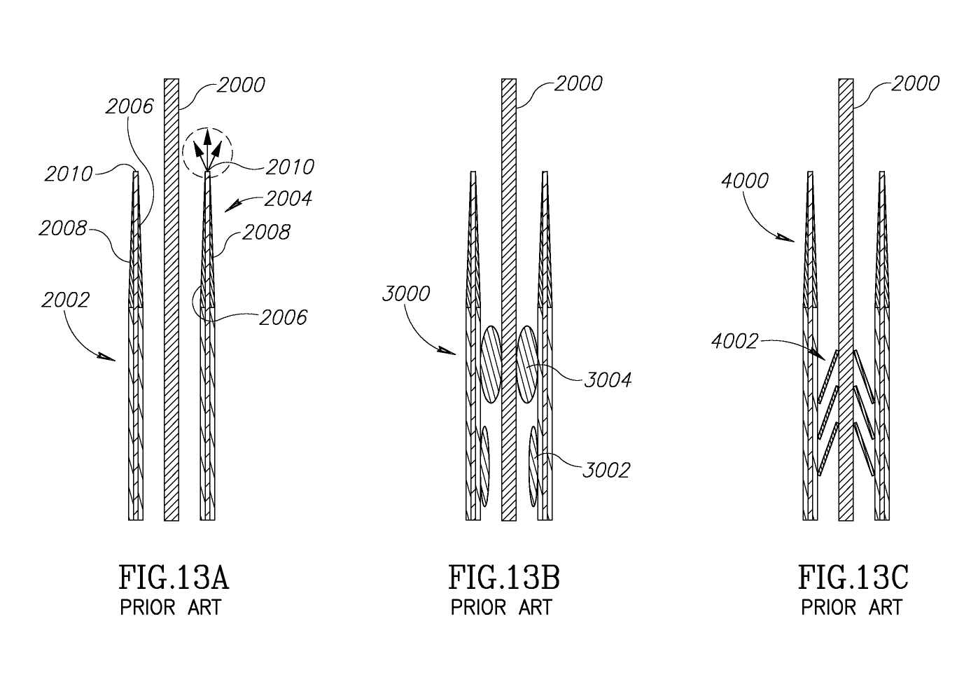

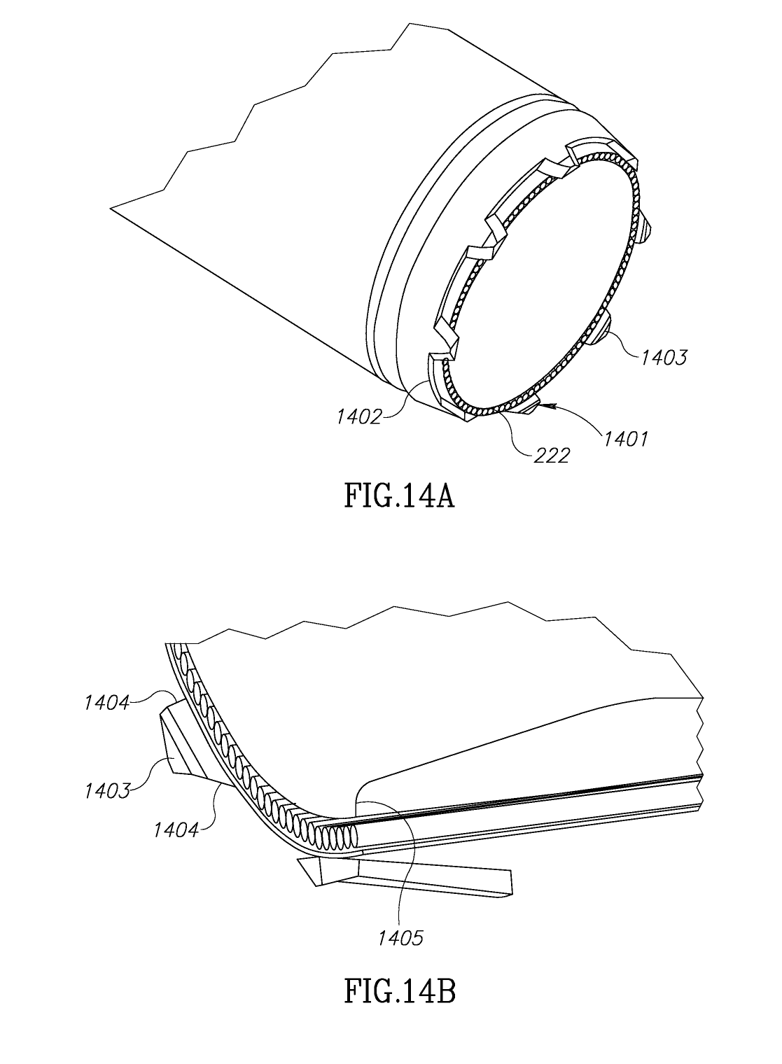

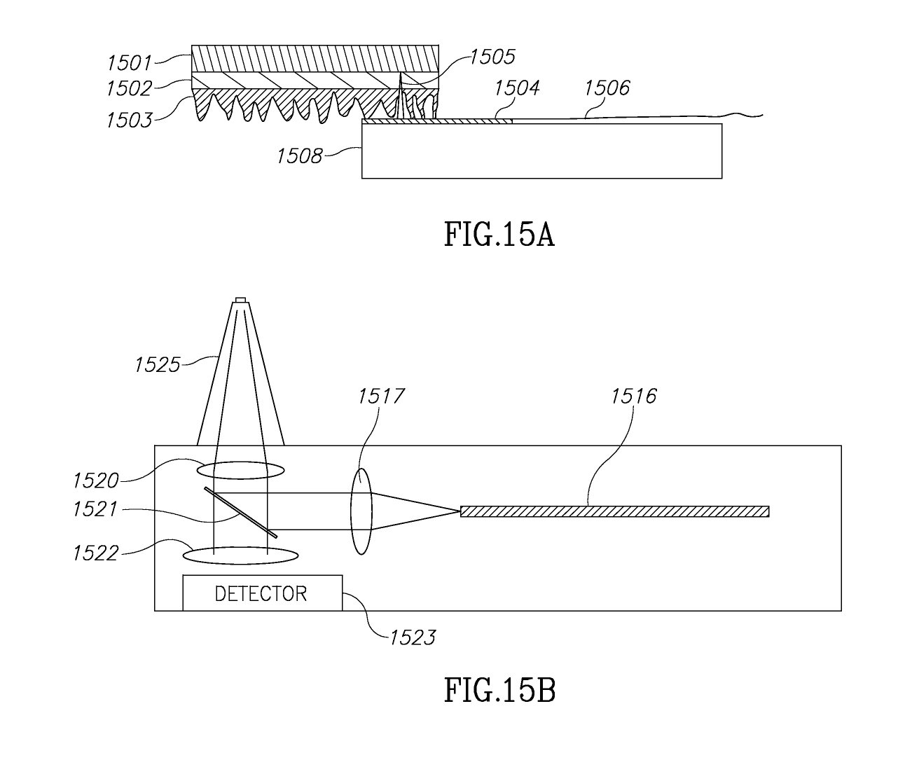

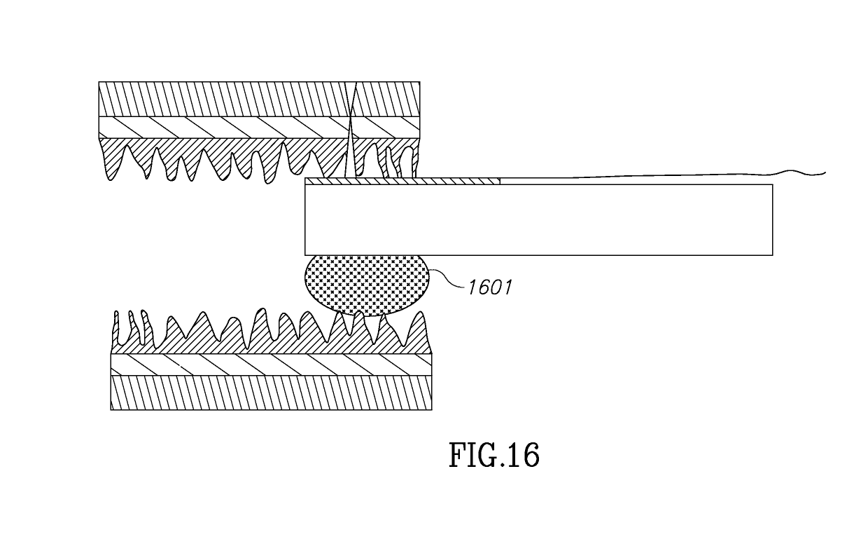

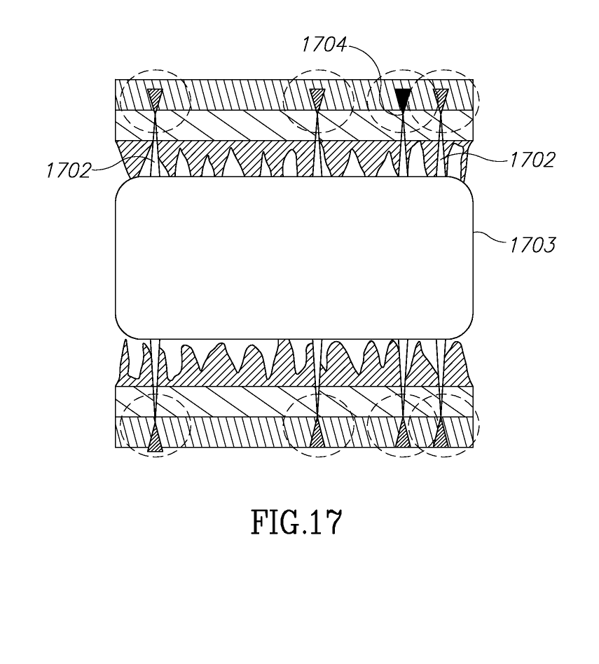

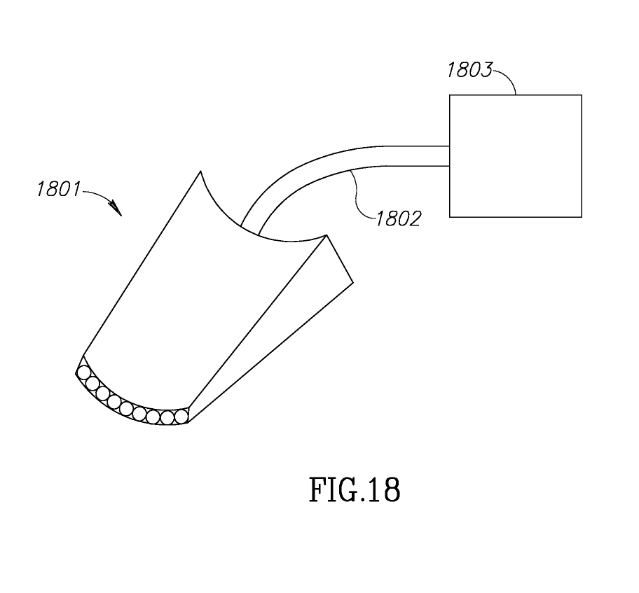

FIG. 10 shows an exemplary tip section of a prior art hybrid device mounted on an endoscope;

FIG. 11A shows a hybrid catheter mounted on an endoscope, during a procedure of detaching undesired tissue;

FIG. 11B illustrates an exemplary mechanical device useful in stretching the tissue in a body lumen such that a flat lesion therein can be effectively treated;

FIG. 12 shows a hybrid catheter assembled on a commercially-available endoscope;

FIGS. 13A-13C show a cross section of a prior art hybrid catheter over a lead to be extracted;

FIGS. 14A and 14B are schematic illustrations of the tip of a novel hybrid catheter, having protrusions on its distal end wall, the protrusions having a blunt distal face, and sharp sideways directed edges;

FIGS. 15A and 15B are schematic drawings of an exemplary catheter assembled on an endoscope, that includes a tip for generating an incision in the duodenum sub-mucosal layer, with an option to include optical based modulation in the muscularis layer;

FIG. 16 is a schematic drawing of an exemplary catheter with the addition of a mechanical element to hold the endoscope in place and attach it to the wall;

FIG. 17 is schematic drawing of an exemplary catheter that is based is based on a stand-alone catheter position in the targeted lumen and includes an array of blunt blades that are functional when energy is applied/delivered through the blades; and

FIG. 18 is a schematic drawing of an exemplary catheter useful for nail fungus treatment.

### DETAILED DESCRIPTION

The prior art shows hybrid catheters and methods for using the same in endoluminal interventions. For example, they may be useful in Barrett's Esophagus management, gastroenterology—such as for removal of sessile and flat lesions in the GI track, and in analogous applications requiring removal of tissue from the inner walls in gynecology and urology interventions. Another example is various vascular applications, such as atherectomy, angioplasty, debulking of plaque in in-stent restenosis, leads extraction, thrombectomy in chronic peripheral and coronary artery diseases and for management of acute blockage of vessels in coronary and neurovascular applications.

The hybrid catheter may be based on a combination of laser and mechanical removal (also “debulking”) of an undesired material from a bodily lumen. In vascular interventions, the catheter may be configured to weaken and/or even cut and detach undesired material with a laser and then, even in cases where the plaque material was not entirely removed, detaching the rest of the plaque material by mechanical means, such as using a blade. The laser may change the mechanical characteristics of tissue, and thereby improve performance of mechanical tools such as various types of blades or shavers. By way of example, the laser may make a soft tissue crispier so it can be effectively crushed using the mechanical tool.

Advantageously, usage of such catheters may obviate the need to photo-ablate (evaporate) most or all of the undesired material. Accordingly, the process may be faster and result in lesser by-products than in common laser ablation, lesser associated mechanical stress and lesser other side effects such as thermal injury resulting from photo ablation. The process may allow using smaller lasers wherein energy is focused at a smaller area and wherein mechanical tools remove traces remaining in the treated area and facilitate further penetration of the laser beam to proceed in effective ablation. In addition, challenging calcified tissue may be successfully treated, despite the difficulty in many of today's common mechanical or excimer lasers to delicately detach such tissue from the vessel's walls. Such catheters advantageously provide for controlled cutting of plaque with minimal or no damage to the vessel's walls.

Such hybrid catheters may be used (for example in atherectomy) alone and/or in conjunction with low pressure balloon angioplasty, stenting, for treating in-stent restenosis with no damage to the stent, and/or for treatment of acute blockages due to plaques and or thrombus (thrombectomy).

The terms “cut”, “dissect”, “resect”, “detach”, “debulk” and “remove” may be used here interchangeably.

The catheters may comprise a tip section, which may be essentially in a cylindrical shape, having circumferentially-directed laser optics, optionally in the form of one or more optical fibers, configured to deliver laser radiation, and a circular-action cutter including one or more blades configured to assist in cutting and/or detaching undesired materials (also “deposits”) from an inner surface of a blood vessel. The one or more optical fibers may be circumferentially-directed, namely, they may be located along an inner surface of the cylindrical tip section, which is near the periphery of the tip section.

Alternatively, the circumferentially-directed optical fibers may be located elsewhere but directed, by way of orientation and/or optical focusing, to radiate an area in front of the circumference of the tip section.

The circular-action cutter may be located in a central part of the tip section, for example, surrounded by the optical fibers. Alternatively, the circular-action cutter may be located in the periphery of the tip section and the one or more optical fibers are located in a central part of the tip section, for example, surrounded by blades.

The one or more optical fibers and the one or more blades may be located in the periphery of the tip section.

The one or more optical fibers and the one or more blades are located in a central part of the tip section.

The circular-action cutter lays on a spring so that a maximum force applied by the cutter is predetermined in order to avoid potential damage, yet be effective. The tip section may include an inner channel maintained at a relative low pressure to suck the undesired material which may be plaque, thrombus material, debris, saline solution used for cleaning and/or the like.

Optionally, a motor is provided to rotate the circular-action cutter in order to improve fragment cutting and/or detaching. Additionally or alternatively, the motor or a different motor may be used to rapidly vibrate the circular-action cutter in order to improve fragment cutting and/or detaching.

Optionally, the circular-action cutter is heated to improve its performance. This may be done by an external heat source, electrical means and/or by the laser radiation.

The catheter tip may be expandable, such that its diameter may be increased after its introduction into the vessel.

The catheter tip may include means for deflection, such that effective working area will be larger than the catheter diameter and enable off-axis work.

The catheter may be useful in cases of Chronic Total Occlusions (CTO), where a guidewire cannot normally be used to pass lesions totally blocking the vessel, and therefore atherectomy is often not feasible, since usage of a guidewire often dictates a certain relative position, and angle in particular, of the catheter's tip section versus the vessel.

An example of an appropriate laser of some embodiments is a solid state ultraviolet (UV) laser emitting pulses in approximately 355 nm and/or 266 nm. An example of an appropriate laser is the Qauntel CFR400, emitting 50 mJ, 10 ns pulses of 355 nm at 50 Hz and/or 40 mJ of 266 nm at 40 Hz. Another example is an Excimer laser.

Another example of appropriate laser can be a 2.9 micron laser with strong absorption if water that can be used directly or by generation of water jet that brakes the plaque. A potential advantage of using a water based jet based effect is that the hard plaque can be broken to enable the catheter, blunt blade to proceed while the relatively flexible walls would be less affected due to their compliance to such mechanical forces thereby enable maintaining a good efficacy safety balance.

A hybrid catheter using a 2.9 micron wavelength laser beam, which is strongly absorbed by water, can be used in more general applications, in order to cut and resect even hard tissue. The impingement of the laser beam on the water, which can be supplied by irrigation passages at the distal tip of the hybrid catheter device, as shown for instance as item 231 in FIGS. 2B to 2D, and as item 477 in FIG. 4C, generates strong jets of water, which can penetrate even hard tissue. This action can be used for resecting nail tissue or even calcified lesions, which are otherwise difficult to remove. In the latter case, the hard lesion may be broken up by the water jets, while the bodily vessel in which the lesion is formed, being flexible, may be reasonably unharmed by the water jets.

In case of using significantly high repetition rates, thermal effects in the tissue may become a problem. This can be at least partially resolved by minimizing ablation area (depth and width), use of short laser pulses and with saline flushing. Another option includes sequential illumination of fibers in a manner that not all the fibers are exposed to laser ration simultaneously, in order to enable thermal relaxation of the affected tissue.

Dyes or substrates may be used to enhance absorption at certain wavelengths, such as 355 nm. For example, sensitization with haematoporphrin or tetracycline prior to the procedure, in order to enhance ablation of the pretreated atheromatous plaque but not insensitised or normal arterial wall.

Another prior art example is a laser emitting pulsed radiation in the mid-infrared (IR) region, such as in the range of 2.8-3 micrometers, a range where water is very effectively absorbed. Additionally or alternatively, radiation at around 2 microns may be used, with a preference for thulium laser emitting at 1910-1940 nm range wherein there is higher absorption of water preferably combined with Q-switched modulation wherein ablation is more effective and reduces lateral damage. For 3 micron emission, an Er:YAG may be used, or another source such as a Mid-IR Holmium Fiber Laser Directly Pumped with Diode Laser that emits at 2840 nm using fluoride fibers [see Optics Letters, Sep. 1, 2007, pp. 2496-2498].

Yet another prior art example is usage of a third harmonic of a Nd:YAG laser at 355 nm, preferably a compact, all solid state, diode pumped laser. The 355 nm radiation usually has a deeper penetration capability compared to the 308 nm radiation, in the depth range of 100 micron or more in relevant tissues and materials. Optionally, very short pulse widths (such as <10 ns) are used, in order to obtain a higher power density, and, in particular, to be able to ablate calcified plaques. In accordance with some embodiments, the energy per pulse is in the range of 10-100 mJ and the pulse frequency is in the range of 10-100 Hz. Optionally, the area of ablation may be flushed with a saline solution in order to reduce side effects (such as cavitation), clean the area of ablation and catheter and/or facilitate collection of debris.

One of the advantages of using 355 nm radiation is that is considered relatively nonmutagenic. The 308 nm radiation of the xenon chloride laser is in the UVB range, which is known to have mutagenic risks. [Walter Alexander. Journal of Clinical Laser Medicine & Surgery. August 1991, 9(4): 238-241. doi:10.1089/clm.1991.9.238.]

Some prior art studies have indicated that third harmonic lasers are generally less suitable to endovascular interventions than 308 nm lasers, due to longer penetration rates and reduced effectiveness of ablation (see, for example, Grundfest W S et al., Am J Surg. 1985 August; 150(2):220-6; and Frank Laidback et al., Lasers in Surgery and Medicine 8:60-65 (1988)). The present embodiments, however, may successfully utilize third harmonic Nd:YAG lasers instead of complex and expensive Excimer lasers. The present embodiments address several problems. For example, in some embodiments, it may not be necessary to laser-ablate all the material whose removal is desired, but rather the laser and the mechanical cutter may share the task; the laser may ablate and/or weaken at least some of the material, while the mechanical cutter completes the job by finally detaching the material from the walls.

A laser that emits radiation in 266 nm may be used. This wavelength has a shorter penetration rate in addition use of compact Excimer laser emitting radiation at 308 nm, as currently used, can be utilized with the current embodiments. According to some embodiments, a system may include means that enable an operator to switch between 266 nm and 355 nm, generated from the same Nd:YAG laser, and means to control power, repetition rate and/or exposure/illumination of specific fiber groups.

An alternative embodiment has replaced UV lasers with a laser with radiation in the 2 micron or 2.8-3 microns, in which ablation is very effective.

Holmium lasers are conventionally used for 2 microns but Thulium lasers have a stronger water absorption and smaller absorption length, which makes them especially suitable for some embodiments. For example, pulsed fiber thulium laser is used. Alternatively, a solid state laser may be used in order to increase pulse power per pulse, which is currently limited in fiber lasers and in view of the limited pulse rate that can be used in order to minimize heat accumulation and damage.

Laser in 2.8-3 micrometer may be Er:YAG. Er:YAG Q-switched are available with pulses in the hundreds of nanosecond range, which may be suitable for present embodiments. See, for example, M. Skorczakowski, et al, Laser Physics Letters Volume 7, Issue 7, pages 498-504, July 2010. Another laser example which may be suitable for specific embodiments is Pantec's model DPM-15 solid state laser, emitting microsecond pulses in the mJ range at hundred of KHz.

Fiber lasers which may be directly diode-pumped, such as a Mid-IR Holmium Fiber Laser, are used. This laser may be pumped from ground level (⁵I8) to an excited energy band (⁵I6) with radiation at about 1150 nm, and the relaxation bands may lead to emission at 2840 nm (relaxation to band ⁵I7) and 2100 nm in relaxation to ground state. Accordingly, this laser may be directly pumped with recently developed high-power, high-brightness diode lasers based on highly strained InGaAs quantum wells that produce output at 1148 nm. See Optics Letters, Sep. 1, 2007, pp. 2496-2498 and Stuart D. Jackson Optics Letters, Vol. 34, Issue 15, pp. 2327-2329 (2009).

The laser may be selected according to the selected resonator optics, for example fluoride fiber lasers to emit laser radiation on the 2.9-μm transition (5I6 to 5I7) and silica fiber lasers to emit radiation on the 2.1-μm transitions (5I7 to 5I8). An advantage of an embodiment using a laser in the region of 2.9-3 micron is that the absorption is very high and results in very short length of absorption, in the order of 15 microns only. Therefore, the relaxation time is shorter so the pulse rate may be increased to above 100 Hz in order to accelerate the procedure.

In addition to the laser beam that interacts with the undesired material, a laser with controlled pulse rate and/or power may be used to interact with the liquid between the fiber tip (exit of laser beam) and tissue, either to allow for “opening” of a passage for the beam (e.g., a channel wherein light is not absorbed when UV radiation is used) to the tissue prior and adjunctive to the required interaction with the tissue, and/or to facilitate the process (when mid-IR radiation is used) benefiting from the “water spray” effect. By way of clarification the tip can be in mechanical contact with the tissue being ablated or not.

Reference is now made to FIGS. 1A, 1B and 1C, which show a prior art cylindrical tip section 100 of a hybrid catheter in perspective, front and cross-section views, respectively, in accordance with an exemplary embodiment. The remainder of the catheter's shaft (not shown) may, in some embodiments, be biocompatible polymer tubing, optionally coated, to minimize friction with the vessel's walls.

Tip section 100 is positioned at the distal end of the hybrid catheter, the end which is inserted into the blood vessel. Tip section 100 may include a housing 102, for example a cylindrical one, at least one optic fiber(s) 104 positioned along an inner surface of housing 102, and a circular-action cutter (or simply “cutter”) 106 positioned inwardly of the optic fibers. Alternatively, (not shown) the circular-action cutter may be positioned outwardly of the optic fibers. It is intended that the following description of the embodiments in which the circular-action cutter is positioned inwardly, be applied, mutatis mutandis, to the alternative, not-shown embodiment. Optionally, optic fiber(s) 104 are delimited and/or supported by a first inner wall 108. Further optionally, cutter 106 is delimited and/or supported by a second inner wall 110.

The catheter may be used with a standard guidewire.

The catheter may be connected to a suction pump that generates low pressure to collect undesired material, saline and/or the like through the catheter. The pump may be a peristaltic pump, which mounts externally to the fluid path, to avoid any contamination of the pump. Optionally, this obviates the need to use disposable parts.

Optic fibers 104, serving as the laser optics of the present hybrid catheter, may be connected, at their proximal end (not shown) to a laser source characterized by one or more of the parameters laid out above. Optic fibers 104 may deliver the laser beams from the source towards the intervention site in the body. In tip section 100 of FIG. 1C, optic fibers 104 are shown as they emit laser towards undesired material 114. One or more areas 116 in undesired material 114 may consequently be modified or even ablated by the laser. Then, cutter 106 may more readily cut into undesired material 114 and detach at least a part of it from the vessel's walls 118.

Cutter 106 is optionally an annular blade extending to a certain depth inside tip section 100 and coupled to a suitable motor (not shown), located in the tip section or further in the shaft, supplying rotary and/or vibratory power to the blade. Optionally, one or more flexible members, such as a spring 112, may interact with cutter 106 at its base, to allow it to retract and protrude from housing 102. Tip section 100 of FIGS. 1A-C is shown with cutter 106 in its protruding position, while tip section 100b of FIG. 1C is shown with the cutter, now marked 106b, in its retracted position. The length of protrusion out of housing 102 may be, for example, up to about 350 microns when treating blood vessels. When protruding, cutter 106 is used for detaching undesired material (also “deposit”) 114 from an inner surface 118 of a blood vessel 120. According to some embodiments, when a certain force (for example, above a predetermined value) is applied to cutter 106 from the front, which may be a result of blockage in blood vessel 120, the cutter may shift its position and retract into housing 102.

The annular blade of cutter 106 may have sufficiently thin edges, such as around 100 microns. Suitable blades may be tailor-made by companies such as MDC Doctor Blades, Crescent and UKAM. The blade may optionally be mounted at the end of a rotatable tube rotated. Such tubes are available from manufacturers such as Pilling, offering a line of laser instrumentation and blade manufacture. The blade may be metal or manufactured by molding a material such as plastic which is optionally coated with a coating having proper characteristics for in-vivo use.

An exemplary tip section may have an external diameter of approximately 5 mm, an internal diameter (within the innermost layer, be it the cutter or an extra wall) of approximately 3.4 mm, and optical fibers each having an approximately 0.1-0.2 mm diameter.

Reference is now made to FIG. 2A, which shows a tip section 200 of a hybrid catheter, which may be similar to tip section 100 of FIG. 1 with one or more alterations: First, one or more fibers 222 of the optical fibers existing in tip section 200 may be used for imaging the lumen of a blood vessel 220 by transporting reflected and scattered light from inside the lumen to an external viewing and/or analysis device (not shown) located externally to the body. This may aid in avoiding perforation of vessel 220 and allowing for on-line monitoring of the intervention process. Second, tip section 200 may be maneuverable, so as to allow different viewing angles and/or in order to align the laser beams and a cutter 206 differently. Third, a cleaning channel (not shown) may be present inside tip section 200 and extending outside the body, through which channel suction 224 is applied in order to evacuate debris of the undesired material which were treated by the lasers and/or cutter 206. Similar embodiments can be used for resection of lesions and controlled incisions in other applications to enable imaged guided procedure. These optional alternations are now discussed in greater detail:

A conventional manner for detection of plaque and other lesions and for monitoring of vessel treatment is based on ultrasound and fluoroscopy. Here, however, one or more fibers 222 may be utilized for detection of lesions and/or to monitor the intervention process on-line, based on the reflection and/or scattering of the laser light from the vessel and/or the deposits. Alternatively or additionally, a different source of illumination may be used, such as through one or more other fibers. The captured light may be transmitted to a sensor such as a CCD, a CMOS or a MOS. The sensing may include a filter or means for spectral imaging, to gain information about the material characteristics (plaque, tissue, calcified plaque, blood clot, etc.). This may enable a quick and effective procedure with minimal risk of perforation, and may enable debulking procedures wherein a guidewire cannot or should not be used.

According to one implementation of the hybrid catheters described in the present disclosure, the photo-acoustic effect is used to obtain image information regarding the position of the catheter tip, and information on structures or layers deeper from the surface than is obtainable by optical imaging. A penetration of 1 mm. or more may be readily achieved. This enables the physician to plan the advancement of the tip more safely than could be done using optical imaging, and to thus avoid perforation or damage to nerves, blood vessels and to enable the detection of boundaries for optimized resection or incisions. This increases the safety and control of the procedure.

The pulsed laser used for ablation, such as at 335 nm, or a weaker pulse, or pulses at another wavelength such as 532 or 1064 nm may be used to generate acoustic waves that depend on the absorption of the laser light and the mechanical characteristics of the tissue. The acoustic waves may be monitored by one or more acoustic transducers that could be for example, piezoelectric or capacitive micromachined ultrasonic transducers, such as are shown in http://www-kyg.stanford.edu/khuriyakub/opencms/en/research/cmuts/general/index.html, which can be embedded in the catheter tip. The transducer can be a single element or an array of elements to create an image. The acoustic transducers may optionally operate in the 1-100 MHz range to provide high spatial resolution. The transducer element can be of size ranging from <100 micrometer to a few mm and in some embodiments can be based on miniature IVUS sensor design for ˜1 mm catheters. Placement of such sensors is illustrated in FIGS. 2B to 2D, which are variously oriented views, FIG. 2B being an end view, and FIGS. 2C and 2D being cut-away isometric views, showing the sensor or sensors 230 placed very close to the distal end of the catheter or in contact with the tip. In order to enable assembly in a small profile catheter tip, elements such as acoustic waveguides or materials are selected for facilitating transmission and decreasing reflection (due to impedance mismatch) of acoustic waves inside the catheter until they get to the transducer. Components and elements for so doing are known in the art, such as at: http://www.ndt-ed.org/EducationResources/CommunityCollege/Ultrasonics/Physics/reflectiontransmissio n.htm, or at http://www.ndt.net/article/jae/papers/22-264.pdf, or at http://arxiv.org/ftp/arxiv/papers/1307/1307.5166.pdf) can be used. The tip may also incorporate saline irrigation paths that can improve acoustic wave coupling, in addition to cleaning of the optical path and fiber optic edge. Exemplary irrigation openings 231 are also shown in FIGS. 2B to 2D, and can be located in between the optical fibers 222 as shown, or directly installed in the walls (not shown in the drawings).

In a similar manner, if instead of the use of laser energy to generate the incision through which the blade penetrates, electrical energy is used, then thermo-acoustic imaging, based on the principle of the thermo-acoustic effect may be used instead of photo-acoustic energy.

The angle of tip section 200 may be controlled to enable by means of tip deflection, enabling removal of material in a cross-section larger than the catheter size. This may be done by mechanical means, such as by selective inflation and deflation of at least two balloons (not shown) attached to the tip section externally at different angles, or a balloon with different compartments 226a-d. Another example is usage of links forming a joint 228, controllable from outside the body using one or more wires (not shown).

The laser optics will now be discussed in greater detail. The laser beam may be directed through fibers each having a core diameter optionally in the range of 40-250 microns. In a configuration where the catheter's circumference is, for example, 15 mm, using fibers with an outer diameter of 50 microns will result in using approximately 300 fibers with a cross-section area smaller than 1 mm², so that for a coupling efficiency of 75%, the energy at the exit of each fiber will be close to 40 mj/mm when pumped with a 50 mJ laser. Adequate fibers for some embodiments may be all-silica fibers with a pure silica core. These fibers can usually withstand about 5 J/cm² in the input. Some embodiments include fibers with a numerical aperture (NA) in the range of 0.12-0.22. An example of a relevant fiber is FiberTech Optica's SUV100/110AN fiber for UV application and the low OH version SIR100/140AN for use with laser in the 1900-2100 nm range or Infrared Fiber Systems, IR Photonics and A.R.T. Photonics GmbH fibers for transmission of radiation in the 2900-3000 range. Embodiments of single mode or multimode may be realized while preservation of beam quality is important but not mandatory in certain embodiments. Some embodiments may include microlenses at the tip area to manipulate the beam at each individual fiber exit.

The power required for effective ablation with 355 nm, 10 nsec pulses (approximately 30-60 mJj/mm²) is close to the damage threshold of certain fibers or above it, which lead, in existing products, to the need of extended pulse length, for example. High peak power may be maintained and accordingly the catheter may include means for delivery of the laser power through relatively bigger optical fibers, e.g. 100 or even 300 micron fibers that do not extend all the way to the end of the tip section, as schematically illustrated in FIGS. 3A-3E.

FIG. 3A shows a prior art tip section 300 which includes a hollow reflective light concentrator 304a with a straight profile or a concave profile (as shown), used to concentrate light from at least two fibers (shown jointly at 304). Hollow concentrator 304a may have metal-based or dielectric coating. Hollow concentrator 304a may form a ring shape surrounding a cutter 306, in manner that radiation from all the fibers is delivered with one concentrator, so that a relatively uniform ring of pulsed radiation is generated at the exit. The exit may include a window (not shown in the figure). Optionally, the optical path may be maintained clean with flushing of saline. Flushing may be through an opening in the front or from the side, between the catheter and an extra lumen that can also facilitate catheter movement in the vessel or in certain embodiments through the central lumen.

FIG. 3B shows a prior art tip section 330 which includes a solid-state light concentrating waveguide 334a for concentrating light from at least two fibers (shown jointly at 304). Solid-state waveguide 334a may be constructed, for example, of Silica with a reflective coating, or a combination of two materials such as Silica and Fluoride-doped Silica.

Solid-state waveguide 334a may be optically coated at the interface with the fiber(s), to improve optical throughput from the fiber(s) to the concentrator. Alternatively, the two may be welded.

FIGS. 3C-3E illustrate the usage of tapered fibers, such as those available from Oxford Electronics of Hampshire, UK. The fibers may be thick 340 at the proximal end of the catheter's shaft, and thin 342 at its distal end—as seen in cross-section. FIG. 3E shows a single tapered fiber 340a in perspective view.

Reference is now made to FIGS. 3F to 3H, which illustrates schematically three views of a hybrid catheter of the type described hereinabove, including lenses 350 positioned at the end of the fibers of the catheter to focus the light emitted from the fibers 351 in at least one dimension, thus creating a line of illumination (continuous or an array of spots) which may be thinner than the width of the fiber core. FIG. 3F is an external view, FIG. 3G is a cut-away view and FIG. 3H is an expanded view of the fiber end. This embodiment allows the achievement of higher flux levels, which may help in beam homogenization due to some overlap between light emitted from different fibers. It can also mitigate some of the risks associated with working with fibers that are in contact with the tissue, such as cracks and damages in the fibers, and full or partial blocking of light emission by deposits that become stuck to the fiber as a result of contact with tissue or materials scattered as result of the ablation. The catheter may include an asymmetrical blade shape wherein the inner walls are different from the outer walls. The inner wall may incorporate a curved shape tip edge 353 and the outer wall may have a tip with a straight line profile 352. The curved inner wall may prevent the tip from being impeded by the guide wire (in atherectomy and in stent restenosis applications) or by an electrode lead (in lead extraction applications) when negotiating curves. In alternative implementations, the outer wall may have a curved shape to enable lower friction motion against the lumen wall and/or the stent.

The distal fiber end may be protected from photo-degradation or impact that results from contact in tissue, by splitting the total pulse energy into separate pulses. The pulses should be separated by a delay by preferably less than 100 nsec, so that the ablation efficiency in the tissue is not significantly impaired. However, the peak power at the fiber edge is reduced significantly, thus avoiding damage even when the fiber tip is not in perfect conditions. The delay between the pulses can be achieved either by pulsing the laser cavity itself, or alternatively, by splitting the output pulse into separate pulses that are made to travel different optical lengths and are then combined, for example by use of S and P polarization combiner schemes as known in the art, or by any other suitable method.

More than one laser may be used to generate multiple pulses within a single approximately pulse train, typically of the order of 100 nsec, or separately in order to increase the effective pulse rate and speed up resection process. This can also enable the use of several, relatively low cost, self/passive Q-switched Nd:YAG lasers which can produce independently the third harmonic 355 nm, or wherein all laser beams are transferred through one harmonic generator module used to generate the third harmonic from the fundamental 1064 nm beam.

Finally, the use of fiber delivered laser energy leads to a number of problems which may limit the maximum energy that can be input to the devices of the present disclosure. Firstly, it may be advantageous to enhance the uniformity of the laser beam before it is coupled into the bundle of fibers for delivery to the hybrid catheter, in order to reduce the variability of the intensity delivered by different fibers within the bundle. Additionally, it may be necessary to reduce the spatial coherence of the laser beam before it is input to the bundle of fibers, to enable insertion of the highest possible energy optical flux into the fibers and to minimize damages which are associated with coherent light beams. This is illustrated schematically in FIG. 3I, which is a schematic block diagram of the optical delivery system for the hybrid catheters described in the present disclosure. An optical beam scrambling or homogenizing module 361 is inserted between the output of the laser 360 and the fiber bundle 362 feeding the hybrid catheter 363. This module can include one or more optical units, including a refractive or reflective beam homogenizer, a waveguide assembly, a conventional diffuser, a diffractive optics diffuser, a beam shaper, a fly's eye lens array or a micro-lens array, as are known in the art.

Reference is now made to FIG. 4A, which shows another prior art option for a circular-action cutter. The circular-action cutter here may be a rotating blade 406 which is rotatable, for example, using a flexible shaft 460 which centrally rotates a plate 462 peripherally connected to the rotating blade. Flexible shaft 460 may be capable of delivering a limited amount of torque, especially when there is bending in the artery, etc. Common mechanical atherectomy devices sometimes use very high rotation speeds to compensate for that. Present embodiments reduce the need for high moments, as the blade is active in an area which has been prepared, namely—cut or at least modified by the laser. Furthermore, the fact that lower torque and rotating forces are applied to the atheroma/plaques, decreases the radial forces applied to the vessels

Reference is now made to FIG. 4B, which is mostly similar to FIG. 4A, except for the way in which rotating blade 406 is being rotated. Rotating blade 406 may be rotated by a miniature motor 464 and suitable transmission 466. Appropriate miniature motors are available from manufacturers such as Namiki, which developed a 1.5 mm-diameter micro-geared DC motor.

Optionally, rotating blade 406 of FIGS. 4A-4B may have shapes that facilitate collection and/or scraping of debulked material, to facilitate collection of debris, as is illustrated schematically in FIG. 4C. This catheter may include a novel design with two sets of blades; one set 471, which are forwardly oriented, facilitates debulking of plaque atheroma and collection of detached material with optionally a scooping shape; and inner blades 472 that are designed to grab and grind and/or crush the debulked, detached bulk material to facilitate its suction through the lumen on the catheter 474. The front blades 471 can be positioned slightly forwards of the fibers (such as 50 microns protrusion from the front) and may partially block the light emitted from the fiber 475. But as the blades rotate the actual blocking is limited in time. Also the protrusion of the blades is limited to avoid direct contact with the lumen walls or stent in stent restenosis applications, even when negotiating turns. In other implementations, the blades may be proximal or in the same plane as the fiber edges. The catheter may further include passages for saline flushing 477 used to clean the optics and the working area and/or to facilitate collection of material detached from the lumen by the catheter.

In order to enable effective debulking in blood vessels, catheters of different dimensions may be used, for example in the range of 4-22 French (approximately 1.3-7 mm). The use of a larger catheter holds the advantage of enhancing the intervention process, but raises an issue of a large opening required for introduction into the vessel and/or accessibility within the vessel itself. Therefore, according to some embodiments, the diameter of the catheter, at least at its tip section, may be expandable. A first example is shown in FIG. 5, which is a cross-sectional view of an expandable tip section 500. A housing 502 of tip section 500 may be made of a relatively flexible material, compared to the rest the catheter's shaft. When the catheter reaches the debulking site, tip section 500 is expanded, to form an outwardly-tapered shape. This expansion may be achieved by introducing a mechanical element which applies pressure on one or more parts in tip section 500. Fibers 504 that transmit the laser beam, may then be inserted into the catheter's walls. Since when the tip section 500 is expended the distance between the fibers also extends, more fibers may be inserted into the walls. Optionally, the mechanical element introduced to expand tip section 500 includes cutter 506. Optionally, expandable tip section 500 may be used in conjunction with the tip section deflecting means of FIG. 2.

In another prior art embodiment of a catheter with an expandable tip section, materials with shape memory, such as Nickel Titanium (known as Nitinol), may be used. The catheter, or at least its tip section, is compressed before introduction into the body, and naturally returns to its pre-compressed shape after it is introduced to the lumen. Nitinol may be used in a structure of a mesh or a braid, to provide sufficient radial force while enabling contraction with low enough radial forces when the catheter is retracted. Some flexibility may still remain at the tip section, to allow accommodation to the physiological shape of lumen. The tip may also include means for controlled deflection.

In some prior art embodiments, the catheter may perform local delivery of drugs which reduce the incident of restenosis, such as Paclitaxel and its derivatives, or soluble forms such as Coroxane. The drug may remain in the site post-treatment and assist in lumen recovery, while preventing overdosing and systematic effects.

The drug administration following the removal of undesired material from the vessel or stent may be achieved by means such as: (i) spraying of drug from nozzles in the external surface of the catheter, or with a tube that includes an array of nozzles at its end, threaded through a suitable channel in the catheter; (ii) by a roller that “paints” the tissue; (iii) by a drug-coated balloon; (iv) by a balloon that includes means to deliver drug through channels in its wall; (v) brushes in the catheter walls; (vi) tubes with nozzles which may change their direction on the way in and out the material removal site.

To optimize long-term efficacy, some prior art embodiments provide means for deep administration of the drug, to be sustained in the deeper layers of the arterial wall or even in remaining plaque but not in the endothelium, thereby allowing new endothelial cells to grow and re-align the lumen, to inhibit restenosis in deep cell layers after the lumen has been restored and re-endothelialized. This may be accomplished by means such as pressure-controlled drug administration, administration below the surface and/or selection of adequate drug forms.

In order to increase absorption of plaque material, the treatment procedure may include administration of one or more substances that increase absorption of plaque at 335 nm such as treating with tetracycline for which the uptake by plaque is a few times larger than in normal tissue. See, for example, Murphy-Chutorian D, et al, Am J Cardiol. 1985 May 1; 55(11):1293-7.

For blood vessel treatment, it is often desired to administer the drug in the deeper layers of the arterial wall but not in the endothelium, thereby allowing new endothelial cells to grow and re-line the lumen. As a result, the drug continues to inhibit restenosis in deep cell layers after the lumen has been restored and re-endothelialized, while, on the other hand, overdosing and systematic effects are eliminated. In some of the cases some plaque material remains on the vessel's walls or stent and the drug formulation and means of administration should take it in account.

Examples of applicable drugs include: Elutax®, SeQuent®, Cotavance™ with Paccocath® coating technology, TADD (from Caliber Therapeutics, Inc.), Advance® 18PTX®, DIOR®, IN.PACT™ Amphirion, Coroxane and more.

The conventional way to administer these drugs to avoid restenosis is with coated balloons. Alternative drug forms such as Coroxane may be administered via IV promptly after the procedure, but this would not result in local administration. It has been suggested in the literature to perform a two-step process wherein a coated balloon follows atherectomy, but this would result in a more complex and costly procedure that can limit routine clinical use.

FIGS. 6A-B, 7A-B, 8A-B and 9A-B include schematic illustrations of a number of prior art exemplary tip section embodiments suitable for local administration of drugs.

FIGS. 6A-B illustrate a tube 600 introduced through the catheter and ending with an array of nozzles or apertures 602 that spray the drug on demand.

FIGS. 7A-B illustrate the use of a roller 700 to stain the tissue. The catheter may include means to allow the roller to get at least partially inside a groove 702 before the debulking procedure, and exit the groove when needed to transfer the drug to the tissue. Roller 700 may include means to apply pressure to the walls in order to increase drug delivery and/or expand the stent in in-stent restenosis (ISR) applications.

FIGS. 8A-B illustrate apertures 800 built into the catheter's housing, and configured to be opened only when needed.

FIGS. 9A-B illustrate an array of tubes or needles 900a-b which are used to administer the drug in a manner that will increase its sustainability. Means to allow the angle of the tubes relative to the catheter to change before and after the debulking procedure and/or in the way inside and outside from the lumen/stent are provided. The tubes may be facing forward 900a when moving forward and backwards 900b when moving backwards. The tubes are optionally made of a flexible, biocompatible material.

Further examples of drug administration may include: a brush to transfer the drug through nipples in the wall of the catheter; a balloon for administration of drug; a balloon surrounding the catheter and being coated with the drug and inflated after the debulking procedure; a balloon with nipples that are used to administer drug on demand; and a coated balloon inserted through the cleaning channel of the catheter.

The prior art embodiments disclosed herein are brought as examples and can be combined for the purpose of vascular intervention in peripheral, coronary and neurovascular applications in chronic and acute conditions and in other medical applications wherein stents have to clean such as in gastro and urology and in applications wherein lumens have to be created or extended such as Benign Prostatic Hyperplasia.

Another clinical application, according to some embodiments of the present invention, is in removal of undesired tissue from a body cavity during an endoluminal procedure. Such procedures can be performed for example in gynecology, urology and in gastroenterology. Such procedures may include, for example, removal of flat and/or large lesions in the gastrointestinal (GI) track and in management of Barrett's esophagus. The motivation is to remove the undesired pathological tissue with minimal complications (e.g., in case of Barrett's esophagus, without esophageal perforations and strictures). This clinical application may require modified embodiments of the hybrid catheters disclosed herein in accordance with some embodiments. FIG. 10-FIG. 12 illustrate catheters for detaching undesired tissue from an inner wall of a body cavity, for example, but not limited to, Barrett's esophagus management, according to embodiments of the invention.

A prior art embodiment is a hybrid catheter which combines a utility of laser radiation to ablate and cut/detach the undesired pathological tissue or modify its mechanical characteristics and mechanical means such a blade or a sharp edge of a wall of the catheter to complete the detaching. This way, the tissue is resected/dissected using the laser radiation and the blade/wall's edge. Thus the blade/wall's edge does not need to be too sharp and are thus configured to cut the tissue without the risk of potential perforation or damage to the body cavity.

Reference is now made to FIG. 10, which shows an exemplary prior art tip section 1000 of a hybrid device in perspective view, mounted on an endoscope 1500, in accordance with an exemplary embodiment. The remainder of the catheter namely—its shaft (not shown) may, in some embodiments, be biocompatible housing, optionally coated so as to reduce friction with the cavity's wall. Endoscope 1500 may be any commercially available scope having, inter alia, working channel 1502, for insertion of medical tools, water/air injector(s) 1504 for cleaning and insufflation, and illuminators 1506. Endoscope 1500 may also include a camera 1508 including CCD, CMOS or MOS sensors for example and optics.

Tip section 1000 is positioned at the distal end of the hybrid catheter, the end which is inserted into the body cavity such as the esophagus. Tip section 1000 has a shape of a sector of a cylinder and is generally configured to be mounted on top of an endoscope (for example, as used in upper endoscopy or colonoscopy). The shape of tip section 1000 is also configured to match the typical anatomy of the body cavity to which it is intended to be inserted. Of course, the tip section of the hybrid catheter (device) may have other appropriate shapes and forms, and can mounted in certain embodiments on another working tool that is used to manipulate it while the process is monitored with another camera such as in laparoscopic procedures. Tip section 1000 may include two walls, an external wall 1002 and an internal wall 1004. One of the walls (external wall 1002 and an internal wall 1004) or both of them may have sharp distal edges to facilitate cutting through the undesired tissue. One of the walls (external wall 1002 and an internal wall 1004) or both of them may be coated with a material that provides sharper edges. At least one optic fiber(s), typically a plurality of optical fiber(s) 1006 are positioned between external wall 1002 and an internal wall 1004. Alternatively, in an embodiment (not shown), there may exist only one wall and the optic fibers may be located along an internal or an external surface thereof. Alternatively, in an embodiment (not shown), there may exist a cutter (similar to the cutter shown in FIGS. 1A-C only having a shape of a sector of a cylinder). In another embodiment, external wall 1002, an internal wall 1004 and/or a cutter (blade) may have two positions, retracted position and protruded position (configured for cutting).

The external wall 1002, an internal wall 1004 and/or a cutter (blade) are configured (such as by virtue of sharpness) to cut through the undesired tissue and thereby detach at least a part of the undesired tissue from the inner wall of the body cavity. If a blade is present, it may be a rotary-action blade and/or a vibrating blade. According to some embodiments, optical fibers 1006 are configured to transmit laser radiation configured to modify an area of the undesired tissue thereby preparing said area for penetration of external wall 1002, an internal wall 1004 and/or a cutter (blade).

The blade may be mounted in a spring so that when force is applied beyond a certain predetermined level the blade enters into its compartment to a retracted position). Alternatively, in another embodiment the position of the blade may be controlled by a physician. This way, the blade is not sharp enough to cut the tissue without the laser so as to avoid potential perforation. Flushing of saline or another appropriate solution at the edge of the catheter may be used to maintain an optical clean path, remove unnecessary material and reduce potential thermal damage and use a “water spray” effect with mid-IR radiation sources.

Reference is now made to FIG. 11A, which shows a prior art hybrid catheter mounted on an endoscope, during a procedure of detaching/resecting (“peeling”) an undesired tissue, according to some embodiments. Hybrid catheter 1001 includes a tip section 1000, transmitting laser radiation and cutting through the tissue. In this figure, hybrid catheter 1001 is used to remove the Barrett's tissue 1008. The illustration shows that different layers can be targeted and removed. Barrett's tissue 1008 (or any other undesired tissue) is cut by catheter and lifted. The catheter, such as catheter 1001, may be (not necessarily) assembled on a commercially available endoscope, such as endoscope 1600 (FIG. 12). According to some embodiments, the tip section of the hybrid catheter (particularly but not limited to) in interventions in the GI track may be position in predetermined angle versus the scope axis and thereby predetermining the depth of penetration of the tip according to the peeling depth required.

This prior art “peeling”-like mode can be thought of in analogy to a “carpenter's plane” but using a “hybrid blade”. The depth of peeling can be adjusted according to the clinical condition such as the depth for Barrett's removal or required according to the stage of the disease and similarly in flat lesion in other places of the GI track. Accordingly the position of the blade knife can be adjusted as well as the distance between the blade and the plane. The catheter with a hybrid blade can be located at a predetermined angle/position and distance from the plane of the endoscope or another tool used to hold the tip. In this embodiment that catheter can be used to make the initial incision of the tissue as a few laser pulses are used to enable generation of a cut to allow the blade to cut through the required layers and then followed by movement of the catheter with the help of the scoop over the organ in forward or backwards direction according to the position angle of the catheter.

The catheter may include a suction device in order to pull and/or attach the tissue to the catheter to facilitate the resection. A stretch force is generated by the suction between the catheter tip and the tissue when the catheter is moved by the operator. Additionally, the catheter may include mechanical means to stretch the target tissue, in order to enable effective realization of the hybrid concept when the relatively blunt catheter tip is designed to penetrate the target with some mechanical force. Reference is now made to FIG. 11B, which illustrates an exemplary mechanical device useful in stretching the tissue in a body lumen 1100, such that a flat lesion 1101 therein can be effectively treated. The example shown in FIG. 11B is that for the treatment of Barrett's esophagus. Before performing the resection, an expandable stretching frame 1105 is inserted by the physician in its closed form into the subject's esophagus, where it is deployed to its operating dimensions, as shown in FIG. 11B. Deployment of the frame from its closed form can be readily achieved, such as by constructing the frame of a shape memory alloy, such as nitinol. The exemplary frame shown in FIG. 11B comprises two end rings 1106 with cross members 1107 linking the rings at intervals around the rings. The stretching frame 1105 is positioned by the surgeon such that one ring is located distal to the region where the lesion tissue is to be removed and the other proximal thereto. Once the frame has been deployed, it stretches the wall 1108 of the lumen in which it is positioned, such that surgical procedure can be performed on the tightly stretched wall without the wall receding significantly from a surgical instrument pushed against the wall. After deployment, a space is formed in the proximal end ring through which an endoscope carrying the hybrid catheter can be passed to gain access to the region of the lesion to be treated. The hybrid catheter 1110 may be of the flat type described in FIG. 11A for “peeling” or shaving the lesion from the inner surface of the esophagus. The hybrid catheter may be equipped with suction openings 1111 in its bottom surface, the openings being connected to a vacuum line traversing the endoscope 1109 such that on application of suction, the hybrid catheter is pulled into close contact with the surface of the lumen, such that resection of the lesion can be efficiently performed, without damage to the esophageal wall. Although the frame has been described in terms of a surgical procedure on the esophagus, its use is not limited thereto, but can be applied to any bodily location where it is necessary to stretch the organ wall to provide resistance to pressure from the instruments used in the procedure being performed.

An example of a structure for applying pushing forces, as opposed to the above described pulling forces, is shown in the embodiment of FIG. 16 hereinbelow, using a balloon that pushes the tip toward the lumen wall.

The catheter may be inserted through the working channel of a standard endoscope or through a special opening made in a dedicated scope. Some prior art embodiments include using a tip with a memory shape that is contracted for introduction through the working channel and is expanded when it exits the endoscope tip. Such a prior art catheter may be based on use of Nitinol. These embodiments enable the physician to perform a diagnostic procedure and, if a pathology is found, to introduce the resection catheter.

In another prior art embodiment depending on the pathology the laser wavelength can be selected to enable reduced tissue penetration or surface ablation such as in 355 nm or 2.8-3 microns lasers or deeper with the 266 nm laser. For below the surface tissue interaction an embodiment of the invention includes a use of a mid-IR laser which had a longer penetration depth. A Thulium laser (potentially a Thulium fiber laser@ lambda=1908-1940 nm, wherein wavelength is matched according to the embodiment to compensate for water absorption wavelength change depending on temperature) may be used for this application since it has a better matching with water absorption length around 2 microns compared to Holmium:YAG and accordingly penetration depth is limited to a few hundred microns and also pulse rate can be increased comparing to Holmium without thermal damage.

One of the potential advantages in using the prior art “hybrid catheter” for debulking of required tissue from lumens such as in the GI track is the side effect of the laser and this is enhancing homeostasis and avoid bleeding. Depending on the specific laser used the effect may not be sufficient to avoid bleeding and some embodiments may include use of an additional laser for the purpose of hemostasis preferably delivered through the same optical fibers.

In accordance with some embodiments, the catheter is connected to a suction pump that generates low pressure to collect undesired material, saline and/or the like through the catheter. The pump may be a peristaltic pump, which mounts externally to the fluid path, to avoid any contamination of the pump. Optionally, this obviates the need to use disposable parts.

The hybrid catheter blade can also be used for improved biopsy procedures enabling relative large sample to be collected for further histology analysis and thereby decrease sampling errors, which are associated with high risk in patients with BE or in gynecology and urology applications.

The hybrid catheter may further include imaging means to detect the required area that has to be treated and to monitor the process on-line, thereby enabling effective “focal therapy” according to the diseases severity from early stage such as Barrett's esophagus without dysplasia to more advanced disease with minimal complications, as it limits damage to the surrounding healthy tissue and avoid mucosal perforation. Similar considerations may apply in gynecology and urology applications. Means to obtain images of the working area may include, for example, commercial fiberscope such Medit INC F2.4 (2.4 mm 45 degrees FOV, with 30,000 pixels) or Olympus LF-2 (designed for tracheal intubation) that can be inserted into 5 mm tubes and includes a 1.5 mm channel for easier aspiration/instillation of fluids, providing images with 90 degrees field of view from >3 mm so the fiber can be placed accordingly. As disclosed hereinabove, the hybrid catheter may be combined with a commercial endoscope, such as a gastroscope preferably such that has enhanced imaging capabilities such a narrow band imaging (NBI) to detect the pathological areas with higher resolution. For example, an Olympus GIF-H180J model (or equivalent) may be used, which has a 9.9 mm diameter at the distal end so the hybrid catheter can be attached to the walls in a manner that it can be conveniently introduced to the body. This enables four-way angulations (210° up, 90° down, and 100° right/left) a 140° field of view and close-up high resolution image can be obtained as close as 2 mm from the tissue, so the laser blade catheter can be attached accordingly to the tip of the scope (relatively advanced in few mm at the front).

Some prior art hybrid catheters have a tip section having optical fibers for transmitting (pulse) laser radiation and inner and/or outer walls having facets that are sharp enough to complete the cutting and debulking (extracting) of leads initiated by the laser but not sharp enough to work alone in order to maintain the procedure's safety. Using the hybrid catheter allows decreasing the requirements from the laser and thus enables use of small solid state lasers, in such way that when the debulking of the leads is not completed by laser cutting the tissue surrounding the leads is performed mechanically (by sharp wall(s) and/or by a blade).

Reference is now made to FIGS. 13A-C, which show cross section illustrations of three types of prior art hybrid catheters for pacemaker and ICD (Implantable Cardioverter Defibrillator) lead extraction.

FIG. 13A shows a cross section of prior art hybrid catheter 2002 over lead 2000 which is to be extracted. Catheter 2002 has a tip section 2004, typically having a circular cross section. Tip section 2004 comprises an inner wall 2006 and an outer wall 2008, at least one of which having a sharp (for example tapered) distal end which thus function like blades. Optical fiber(s) 2010 are located between inner wall 2006 and outer wall 2008 and are configured to transmit laser radiation through the distal end of tip section 2004 (as marked by the arrows).

The laser radiation modifies (e.g., ablate, partially ablate, weaken, cut, etc.) the tissue surrounding the lead and thereby preparing the tissue for penetration of the sharp distal edge of inner wall 2006 and outer wall 2008, such that walls are configured to cut through the modified tissue and thereby detach lead 2000 from the tissue.

The catheter may include means to hold the lead in order to extract it from the body. These embodiment aim to replace a complicated process known in the art wherein a lead locking device is inserted (e.g. Spectranetics Lead Locking Device (LLD®)) and then another catheter used for laser ablation (Spectranetics SLS® II) is inserted. Two examples of means for holding and retracting the lead are schematically illustrated in FIGS. 13B and 13C, in accordance with some embodiments.

FIG. 13B shows a cross section of prior art hybrid catheter 3000 over lead 2000 which is to be extracted. Catheter 3000 may be similar to catheter 2002, but further includes a “donut shaped” balloon (3002/3004) connected to an inner wall of hybrid catheter 3000. When hybrid catheter 3000 is penetrating through the tissue surroundings lead 2000 the balloon is deflated 3002. When hybrid catheter 3000 is pulled out in order to extract lead 2000 the balloon is inflated (3004) and “holds” lead 2000 and thus assist in its extraction.

FIG. 13C shows a cross section of prior art hybrid catheter 4000 over lead 2000 which is to be extracted. Catheter 4000 may be similar to catheter 2002, but further includes “grabbing elements” 4002, configured to allow smooth penetration of catheter 4000 through the tissue surroundings lead 2000 but to hold lead 2000 in a predetermined force when moving outside. According to some embodiments, the catheter may include means to release this holding in cases there is a need to retract the catheter without the lead

Reference is now made to FIGS. 14A and 14B which show isometric drawings of the tip of a novel hybrid catheter, in that it has additional protrusions or bumps 1401 on its distal end wall 1402 around the fibers 222. FIG. 14A is an overall view, while FIG. 14B is a magnified cut-away view of a portion of the distal end of the catheter. This structure enables the physician to further facilitate separation, without the need to apply high forces, either in pushing the catheter forward, or in pulling back the leads, after or while the laser fires to impact the target. The protrusions have a blunt distal face 1403, such that they are not sharp enough to perforate the tissue when the catheter is moved forward. Tissue penetration is performed not only by means of the laser emission from the fibers, but also, if necessary, by rotation of the catheter in either or both directions, such that the sharp edges 1404 of the blunt protrusions 1401 can cut the tissue during such rotational movements. The catheter may have different flexibility along its length, preferably being more flexible at the distal end to accommodate the shape of the vessels in which it is being used, while the more proximal section is more rigid to enable transmission of rotational and/or pushing forces. In some embodiments the shape of the inner part is different than that of the outer wall to facilitate following turns in the lead, for example at the end of the subclavian vein, avoiding impact on the lead or shaving deposits attached to the lead. This is illustrated in the profile of the inner wall shown in FIG. 14B, which has a rounded end shape 1405 compared to the shape of the outer wall. Improved sliding of the catheter over the lead enables the use of smaller catheters and thereby minimizes undesired impact with the vessel walls that can result in perforation. Procedure safety is thereby increased. In some embodiments, in order to facilitate sliding of the catheter over the lead which can be covered with deposits and have damaged walls, the inner catheter is made from a low friction material such as Teflon or coated with biocompatible hydrophilic coatings. In the example shown in FIGS. 14A and 14B, the protrusions or bumps are incorporated into the outer wall of the catheter, but it is to be understood that they could equally well be incorporated on the inner walls of the catheter. Such an implementation may be of specific interest in applications such as in-stent restenosis to avoid any risk that the protruding elements will hit the stent while the head is rotated. The catheter may include an automatic rotation mechanism, or a vibrating mechanism of the tip.

A further implementation of the hybrid catheters of this disclosure can be configured to “blind” the duodenum to the passage of food and/or modulate physiological response and function. Multiple clinical studies and reports have clearly indicated that bariatric surgery can help reduce weight and obtain complete remission of type 2 diabetes (T2D) or improve disease management including impact on insulin resistance. This is specifically the case in certain procedures such as those that bypass the duodenum, such as RYGB (Roux-en-Y gastric bypass) and & GJB (gastrojejunal bypass) surgery. Unfortunately, surgery is associated with high risk and high cost is not the optimal solution for management of the majority of T2D and non-obese patients estimated at hundreds of millions worldwide.

Previously, attempts to obtain effects similar to surgery albeit with devices have been pursued, which included the use of staplers to reduce stomach size, insertion of devices into the stomach (most common of which is the intra-gastric balloon), implantation of electrical stimulators that intervene with stomach function (gastric electrical stimulation), use of sleeves that bypass the duodenum such as the EndoBarrier® (GI Dynamics™) and RF ablation applied to the surface of the organ in the gastrointestinal —GI tract with non-penetrating electrodes as in US 2008/0275445 A1 to Barxx, ablation of the area around the pyloric sphincter as in EP1567082 A1 to Curon.

Each of these methods suffers from inherent limitations. For example, use of the EndoBarrier® is associated with adverse events and side unwarranted side effects such as those recently published (Verdam F J et al. Obesity 2012, Vol 2012-http://www.hindawi.com/journals/jobes/2012/597871/).

It is an object of the present implementation of such hybrid catheters to overcome the limitations of the prior art and to provide novel minimally invasive solutions for endoluminal interventions that block neurohormonal and others signals triggered by food passing through the GI tract while minimizing side effects by selective spatially localized interventions.

In one embodiment a method and apparatus is described to block neural signals generated when a meal passes through the duodenum by impacting, denervating, modifying, blocking, ablating, damaging, or cutting the plexus that resides in the submucosa of the duodenal wall (the submucosal or Meissner's plexus), which transmit signals acquired through chemical sensors in the duodenum, locally as well as to the Vagal and Ganglia nerves. Another exemplary implementation is a method and apparatus for modifying, impacting, blocking, ablating, damaging, or cutting of the plexus that reside in the tunica muscularis of the duodenal wall (the Auerbach's plexus or Myenteric plexus) to enable blocking of signals from mechano-sensors and other chemo-receptors that pass through the Myenteric plexus. These methods and apparati are thus able to “blind” the duodenum to the meal and GI secretions traversing into the intestine from the stomach. This can modulate/impact metabolic balance and/or motility and/or impact gastrointestinal physiology in line with the “foregut hypothesis” set forth by several publications (such Francesco Rubino, et al, The Mechanism of Diabetes Control After Gastrointestinal Bypass Surgery Reveals a Role of the Proximal Small Intestine in the Pathophysiology of Type 2 Diabetes, Ann Surg. 2006 November; 244(5): 741-749; Pories W J et al., The surgical treatment of type two diabetes mellitus, Surg Clin North Am. 2011 August; 91(4):821-36, Cummings D E, Overduin J, Foster-Schubert K E. Gastric bypass for obesity: mechanisms of weight loss and diabetes resolution. J Clin Endocrinol Metab. 2004; 89:2608-2615), incorporated herein as references, each in their entirety, which proposes that this region of the intestines may play a significant role in the development of T2D when over stimulated with nutrients in susceptible individuals, for example via the induction of a putative signal that promotes insulin resistance and type 2 diabetes.

The blocking of the signals triggered by food either by chemical sensors or mechanical sensors may also modulate motility related functions of the GI tract organs, such as modulation of gastric accommodation and relaxation triggered by meal passage through the duodenum and/or stomach.

The duodenum has several physiological functions and the methods and apparatus described herein aim to spare, preserve, both short and long term impairment of the physiologic function(s) that are not required to be impacted, modulated, or blocked for the management of diabetes, insulin resistance and obesity. In particular, the duodenum villi, bicarbonate secretion, the maintenance of fluid/electrolyte imbalance, etc. and even nutrient absorption do not need to be impaired in order to impact metabolic balances and/or affect motility. The methods and apparatus may be able to selectively and locally modulate signals triggered by food and/or physiological functions associated with food ingestion, with minimal collateral tissue damage and unwarranted side effects.

Interventions can be performed at several locations along the duodenum to impact, block, modulate various pathways and sensors spread across the various layers of the duodenal wall and can be of sufficient density so as to affect various mechanisms involved in T2D & obesity, including but not limited to the signals transmitted. By mode of example, these interventions could:

1. locally modulate neural pathways between the different plexus and sensors spread across the various layers of the duodenal wall and

2. be sufficiently dense or close to each other as to effectively modulate signals transmitted externally from the duodenum through the local bundles that interface with vagal and ganglia nerves. In preferred embodiments of this invention, several places across along the proximal duodenum can be impacted but also more proximal locations such as the distal gastric region and pylorus of the stomach as well as distal-intestinal locations such as the duodenal-jejunal junction and the jejunum can be impacted.

In interventions in the Myentric plexus, motor neurons may be damaged as well. However, the method benefits from the fact that efferent nerves (in contrast to afferent, in most cases) can recover their and have their physiologic function restored.

Such hybrid catheters, their method of use, and apparatus and the application thereof is not limited in its application to the details set forth or contained herein and that the present invention can be modified to modify signals originating from the stomach or other organs/positions along the GI tract.

The present implementations rely on endoscopy-based or stand-alone catheter based interventions to modulate sensory signals and/or alternate sensory pathways and/or physiological functions in the duodenum, stomach and/or other parts of the GI tract.

In some embodiments, nerve signal blocking can be achieved by using mechanical disruption methods, energy or a combination of both to create incisions/dissections in the wall of organs in the GI track.

By mode of examples, a few embodiments are presented to selectively ablate/damage/modulate (with and without on-line monitoring) nerve plexus in the sub mucosal and optionally on the muscle layers, and/or including in the interfaces/connections with vagal and ganglia nerves. A few embodiments using different configurations and energy modalities are disclosed using “blades” with and without on-line feedback. On-line feedback examples based on optical imaging or photoacoustic or thermoacoustic effects were discussed before in the context of resection or debulking or dilation applications and are applicable also in this implementation.

Reference is now made to FIG. 15A, which illustrates a duodenum wall with its three layers—muscularis 1501, sub-mucosal 1502 and mucosal 1503 with its vilae. One exemplary schematic implementation of the apparatus for performing the methods described herein incorporates a catheter 1504 of the type described in this disclosure, with its optical fibers and electrical signal leads 1506 for enabling laser ablation pulses, rotation, signal acquisition to be supplied to the catheter. The catheter is assembled on an endoscope 1508 which is operable to bring the catheter to the region in the duodenum where the treatment is to be performed. The catheter may include blade rotation and/or imaging capabilities, as previously discussed. The catheter is adapted to create thin controlled incisions 1505 in the sub mucosal layer, to cut the sub-mucosal plexus 1502. The doctor uses the “blunt” nature of the catheters of the present disclosure, in order to safely maintain accurate control of the incision depth, alternately using the laser pulses and the mechanical advancement features provided by these catheters combined with real-time imaging to determine when the blade has penetrated to a sufficiently depth, and to then stop catheter advancement to avoid perforation. The blade may conveniently be aligned perpendicularly to the lumen axis while in other embodiments it may be aligned at other angles. Fiber optics are used all the way to the catheter tip while in other embodiments the finals travel to the tip edge is performed with the help of optics that delivers the beam to the edge of the catheter.

Reference is now made to FIG. 15B, which schematically illustrates an enlarged and cut-away schematic illustration of the catheter 1504 of FIG. 15A, in which the laser light is transmitted with a fiber 1516 and is collimated with a lens 1517. The collimated beam may be reflected towards the tissue with a dichroic mirror or a beam splitter 1521, and is focused with a lens 1520 onto the edge of the hybrid blade 1525. An imaging lens 1522 may be used to obtain information about the position of the catheter edge, using back reflected, scattered, or fluorescence light imaged onto a detector array or camera 1523. The fiber may deliver one or more wavelengths, or one or more laser sources may be used if imaging at a different wavelength is of interest. In such embodiments, or where fluorescence is used for imaging, a dichroic mirror can be suitable. If imaging is done at the wavelength used for ablation, a beam splitter may be more appropriate. In other embodiments an acoustic transducer is used to obtain microscopy information based on the acousto-optic effect, as described in relation to FIGS. 2B to 2D. In other implementations, the laser light may be replaced with bipolar-based ablation, where, instead of fibers, there is an array of leads that enables tissue ablation and incisions according to the principle of bipolar blades, such as is provided by the PowerBlade™ as supplied by LiNA Medical of Glostrup, Denmark, but herein assembled as part of a hybrid blade.

A series of dissections (incisions) can be performed across the duodenum. The spacing of such incisions may be varied, as in certain embodiments, there may be need for more closely spaced incisions at the proximal area than in more distal areas, wherein the density of the connection to the vagal and ganglia nerves is lower and the sensory function is different.

The objective is to enable control of the incision depth—generally a maximum of 0.5-1.5 mm from the duodenum mucosal plane—regardless of the inter- and intra-variability of the folded mucosal layer. When required, the plexus in the muscularis layer can be modulated by energy delivered from the tip edge of the catheter, preferably with a mechanical incision to enable the organ to maintain its mechanical strength, avoid deformation or leaking and to return to function. In addition incisions in the jejunum and/or stomach can be performed. The catheter can be optionally inserted through the working channel of the endoscope (not shown) and the endoscope should be configured to enable manipulation, rotation and/or fixing of the catheter relative to the lumen.

The operator can make circular incisions, spirals or other shapes manually, and optionally, the catheter may include mechanisms as known in the art, that enable rotation without the need to rotate endoscope.

In addition to their energy transmission function, the fibers may be optionally used for optical imaging of the progress of the catheter, to avoid crossing the muscle layer and perforating the duodenum or other organ on which the procedure is being performed.

Reference is now made to FIG. 16, which illustrates another exemplary embodiment, similar to that shown in FIG. 15, but including an additional structure to assist in attaching the catheter and/or endoscope to the wall in order to ensure that the tip makes the incision or dissection to the required predetermined depth. In this example, a balloon 1601 is used to enable spatial fixing of the endoscope and catheter relative to the walls of the duodenum. Movement of the assembly along the duodenum to new locations can be achieved by successive deflation and inflation of the balloon. Alternatively the balloon 1601 can be coated with a material such as a hydrophilic coating that allows it slide. The catheter and/or endoscope can be rotated manually or can be rotated automatically in order to make the incision over the entire circumferential wall of the duodenum. In the example illustrated, the fixing element 1601 can be located before or after the rotating tip.

In yet another embodiment, the system can be used to generate incisions on an organ having compliance at a level such that it may move when pushed. The system uses a catheter of the type described in this disclosure with a series of blunt blade assembles, in which mechanical contact with the lumen walls is maintained by use of a balloon lodging the assembly against the wall. Reference is now made to FIG. 17, which illustrates such an exemplary embodiment wherein a stand-alone catheter is positioned in the targeted organ and an array of hybrid blades 1702 of the type described in this disclosure are applied to the lumen walls by an inflatable balloon 1703. The blades are activated to generate the predetermined incisions by applying laser energy through the fibers attached to each of the blades. By this means, incisions may be made at successive locations along the length of the lumen without the need to move the blade assembly, but just by transferring the laser input from one blade to the next sequentially. Thus, in the device shown in FIG. 17, only the blade marked 1704 is being supplied with ablating laser power, as shown by its darker shaded outline, while the other blades are in position along the lumen, ready for their activation by the laser power.

Such an inflatable balloon can be used in any circumstances where it is necessary to tension the wall of the organ or lumen on which a procedure is being performed, in order to maintain the spatial position of the wall of the organ or lumen relative to the catheter. Alternatively, a vacuum sleeve may be used to keep the wall in position against the vacuum sleeve. This type of structure is useful when it is necessary to resect flat lesions, which, were it not for the mechanical tensioning of the surface on which the lesion is situated, would tend to move away from the cutting element as it pushes onto the surface of the organ.

In yet another embodiment, in place of the laser energy, a thin electrode or an array of microelectrodes can be used in conjunction with the blunt blades, that can ablate simultaneously or sequentially in a mono or bipolar manner. This arrangement is able to generate incisions in mucosal and sub mucosal layers with minimal lateral damage. Optical damage of nerves in the muscle layer can be accomplished using the same laser energy or another wavelength such 532 nm or a Holmium/Thulmium laser to obtain a higher penetration depth. In another embodiment, controlled damage in the external muscularis layer is obtained according to the characteristics of the electric field use in the blade.

While the current disclosure focuses on endoluminal applications, it is also useful in other medical applications such as the removal of nails and photo ablation, and the killing of pathogens. Reference is now made to FIG. 18, which illustrates schematically a system for management of nail fungus, incorporating a hybrid catheter of the types described in this disclosure. Such a device has an advantage over prior art laser treatment devices in that the catheter can deliver both a resectioning laser emission in order to remove part of the nail, and a photo-chemical treatment achieved by use of a laser at a different wavelength where the photo-chemical degradation effect is more efficient. Use of the catheter with two applied wavelengths thus enables the results of the treatment to be seen within a short time, of the order of one or two weeks, whereas with the prior art, single laser treatment, where none of the nail is removed, assessment of the success of the treatment must await the growth of new nail tissue, which could take many weeks or even months. Generally, the resecting laser operates with the assistance of the mechanical cutters described in this disclosure, although it may be feasible that the device has only the two laser ablation systems, if efficient focusing methods for the resecting laser are provided,

The catheter has a narrow blunt tip 1801 through which laser radiation is delivered by an array of fibers assembled in a bundle 1802 that delivers the energy from one or more lasers 1803. The laser wavelengths are selected to perform any of resection, removal, photo treatment or ablation.

Different wavelengths can be used for resection and for photo-chemical treatment. This has the advantage that the most suitable wavelength for each part of the treatment may be used. Thus in one exemplary implementation, mid-IR radiation at 2.9 microns is used in the resection process due to its strong superficial absorption. Alternatively, the resection can be performed by an ablation process at 355 nm. After the nail is resected, a second wavelength with deeper depth of absorption is used, such as 1.064 microns from a Nd:YAG laser, which is more efficient for performing the photochemical treatment which destroys the fungus. The 1.064 micron radiation is able to penetrate more than superficially, even to a depth of more than 1 mm, such that the fungicidal effect of the laser is more effective, operating both within the depth of the nail. The advantage of this device and method over prior art laser nail treatments is that the device is able to resect and photo-treat the nail in one procedure, even by simultaneous application of both laser wavelengths simultaneously. Alternatively, a wavelength shifting method can be used to the spectral region of strong absorption of water to enable deeper penetration. In another embodiment, a water jet triggered by a laser at 2.9 micron can be used.

In another embodiment, instead of using a laser to create a gap through which the catheter edge “blade” is advanced, an electrical discharge current can be delivered from RF micro electrodes arranged in an array across the blade, and activated simultaneously or sequentially to create a channel through which the mechanical blade can enter.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

While the invention has been specifically described in connection with certain specific embodiments thereof, it is to be understood that this is by way of illustration and not of limitation, and that various changes and modifications in form and details may be made thereto, and the scope of the appended claims should be construed as broadly as the prior art will permit.

The description of the invention is merely exemplary in nature, and, thus, variations that do not depart from the gist of the invention are intended to be within the scope of the invention. Such variations are not to be regarded as a departure from the spirit and scope of the invention.

In the description and claims of the application, each of the words “comprise” “include” and “have”, and forms thereof, are not necessarily limited to members in a list with which the words may be associated.

In addition, where there are inconsistencies between this application and any document referenced or incorporated by reference, it is hereby intended that the present application controls.

1. A device for performing cutting action on a region of a tissue, comprising:
an applicator adapted to direct energy from the distal end of said device onto said region of said tissue;
an element for performing mechanical cutting, said element being aligned such that said mechanical cutting is performed in the same regions of said tissue where said energy is applied; and
at least one acoustic sensor disposed in said distal region of said device, wherein when said energy is pulsed, said at least one acoustic sensor is configured to perform imaging of said tissue by detecting acoustic waves generated therein by impingement of said pulsed energy in said tissue.

2. The device according to claim 1 wherein said energy is laser energy, and said acoustic waves are generated by a photo-acoustic effect.

3. The device according to claim 1 wherein said energy is RF electrical discharge energy, and said acoustic waves are generated by the thermo-acoustic effect.

4. The device according to claim 2 wherein said applicator is an array of optical fibers adapted to direct said pulsed laser energy onto said region of said tissue.

5. The device according to claim 4 wherein said optical fibers are arranged in a circular disposition, and said element for performing mechanical cutting is a circular cutter co-axially aligned with said optical fibers.

6. The device according to claim 1, wherein at least one of said acoustic sensors is an ultrasound transducer.

7. The device according to claim 1, wherein said pulsed energy is also used to degrade said tissue.

8. The device according to claim 1, wherein a separate source of pulsed energy is used to degrade said tissue.

9. The device according to claim 1, wherein said at least one acoustic sensor is an array of acoustic sensors.

10. A device for performing cutting action on a region of a tissue, comprising:
a circular array of optical fibers disposed at the distal end of said device such that laser emission from said optical fibers falls on said region of said tissue; and a plurality of protrusions disposed on said distal end of said device, said protrusions having a blunt distal end face, and sharp lateral edges, such that rotation of said device causes said protrusions to generate mechanical cutting action in said region of said tissue.

11. The device according to claim 10 wherein said blunt distal end faces of said protrusions are configured such that they generate resistance to penetration of said device into said tissue when said device is pushed towards said tissue.

12. The device according to claim 10, wherein said lateral edges are sufficiently sharp to cut said tissue when said device is rotated manually.

13. The device according to claim 10, wherein said device has low friction coatings on at least its inner wall, such that it can slide readily into said tissue.

14. The device according to claim 10, wherein said device has a rounded profile to the inner wall of its distal end, such that it can slide readily over a guide wire or a pacemaker lead or a defibrillator lead.

15. The device according to claim 10, wherein said device has a rounded profile to the outer wall of its distal end, such that it can slide over the tissue.

16. A device for performing cutting action on a region of a tissue, comprising:
an applicator adapted to direct energy from the distal end of said device onto said region of said tissue;
an element for performing mechanical cutting, said element being aligned such that said mechanical cutting is performed in the same regions of said tissue where said energy is applied,
wherein said mechanical cutting element comprises a first set of blades for detaching material from said tissue, and a second set of blades for cutting up said detached material to facilitate its removal from said region.

17. The device according to claim 16, wherein at least said second set of blades has a scooped shape for removing said detached material.

18.-33. (canceled)
